(19) United States
(12) Patent Application Publication
(10) Pub. No.: US 2018/0003723 A1
(43) Pub. Date: Jan. 4, 2018

(54) METHODS AND SYSTEMS FOR DIAGNOSING SLEEP DISORDERS

(71) Applicant: OASIS DIAGNOSTICS CORPORATION

(72) Inventors: BALWANT RAI, BHANGU-SIRSA (IN); JASDEEP KAUR, MODEL TOWN-KAPURTHALA (IN)

(21) Appl. No.: 15/510,812

(22) PCT Filed: Sep. 15, 2015
(86) PCT No.: PCT/US15/50282

§ 371 Date: Mar. 13, 2017

(30) Foreign Application Priority Data
Sep. 15, 2014 (IN) 2645/DEL/2014

(51) Int. Cl. G01N 33/68; G01N 21/77; G01N 33/92; G01N 33/64; G01N 33/70

(52) U.S. Cl. CPC G01N 33/6896; G01N 33/64; G01N 33/70; G01N 33/92; G01N 21/77; G01N 2021/7786; G01N 2570/00; G01N 2800/2864; G01N 2800/52; G01N 2021/7759

(57) ABSTRACT

Methods and compositions for risk detection, early diagnosis, prognosis, and monitoring of sleepiness in an individual by measuring the amount of specific biomarkers present in a bodily fluid and comparing them to a reference level of biomarkers in a sample from a healthy person, a person previously diagnosed with sleepiness, or an earlier sample from the individual of interest.

### CROSS-REFERENCE TO RELATED APPLICATIONS

This application claims benefit of Indian Patent Application, Application Number 2645/DEL/2014, filed Sep. 15, 2014.

### BACKGROUND

Sleep is increasingly recognized as important for general health and functioning, with insufficient sleep linked to motor vehicle crashes, industrial disasters, and medical and other occupational errors. (Institute of Medicine. Sleep Disorders and Sleep Deprivation: An Unmet Public Health Problem. Washington, D.C.: The National Academies Press; 2006.) If sleep is cut short, the body fails to complete all of the phases needed for muscle repair, memory consolidation, and release of hormones regulating growth and appetite, resulting in mental as well as physical fatigue.

The National Academy of Sciences estimates that fifty to seventy million Americans suffer from a “chronic disorder of sleep and wakefulness.” (Institute of Medicine. Sleep Disorders and Sleep Deprivation: An Unmet Public Health Problem. Washington, D.C.: The National Academies Press; 2006.) A recent study by the Centers for Disease Control and Prevention revealed that almost five percent of adults acknowledge nodding off at the wheel at least once during the previous month. The U.S. Department of Transportation has determined that driving while drowsy causes forty thousand injuries a year in the United States and more than fifteen hundred deaths (US Department of Transportation, National Highway Traffic Safety Administration, National Center on Sleep Disorders Research, National Heart Lung and Blood Institute. Drowsy driving and automobile crashes [National Highway Traffic Safety Administration Web Site]), with one in every six deadly car crashes resulting from a fatigue-impaired driver.

The International Classification of Sleep Disorders distinguishes more than 80 different sleep disorders affecting approximately 35 to 40% of the U.S. adult population annually. These disorders are a significant cause of morbidity and mortality; however, the prevalence, burden, and management of sleep disorders are often ignored or overlooked by individuals and society in general. Sleep stages and other characteristics of sleep are commonly assessed by polysomnography in a specialized sleep laboratory. Measurements taken include an electroencephalogram (EEG) of brain waves, electrooculography (EOG) of eye movements, and electromyography (EMG) of skeletal muscle activity. These methods are invasive, expensive and time consuming. There is therefore an unmet need for a more time efficient and convenient means to evaluate, detect, diagnose, prognosticate and monitor sleep loss and disturbances.

### SUMMARY

Provided herein is a non-invasive means for diagnosing, measuring and monitoring sleep disorders. As disclosed herein, multiple biomarkers in the bodily fluids of an individual may be quantitatively or qualitatively measured alone or in combination as a diagnostic for sleep disorders. Levels of biomarkers may also be used to monitor the progression and severity of the sleep disorder and determine the effectiveness of a particular treatment in alleviating the sleep disorder.

The methods and kits described herein include the identification of biomarkers such as proteins in a biological fluid, such as saliva. Such biomarkers may be identified by any means generally used by one of skill in the art. In some embodiments, these biomarkers are identified using antibody-based methods, such as, but not limited to, an enzyme-linked immunosorbent assay (ELISA), a radioimmunoassay (RIA), a lateral flow immunoassay, or proteomic approaches that utilize various detection methods.

Biomarkers as used herein may be any one or two or more of, 2-propanediol, 1,5-anhydroglucitol, 2,3-dihydroxyisovalerate, 2′-deoxyguanosine, 2′-deoxyinosine, 2-hydroxy-3-methylvalerate, 2-hydroxybutyrate, 2-hydroxyglutarate, 3-(4-hydroxyphenyl)lactate, 3-(4-hydroxyphenyl)propionate, 3-dehydrocarnitine, 3-hydroxybutyrate, 3-methyl-2-oxobutyrate, 3-methyl-2-oxovalerate, 3-phenylpropionate, 4-hydroxybutyrate, 4-hydroxyphenylacetate, 4-methyl-2-oxopentanoate, 5,6-dihydrothymine, 5-aminovalerate, 5-oxoproline, 8-hydroxyoctanoate, adenine, agmatine, alanine, alanylalanine, alanylisoleucine, alanylleucine, alanylproline, alanylvaline, allantoin, alpha-glutamylglutamate, alpha-hydroxyisocaproate, alpha-hydroxyisovalerate, alpha-ketoglutarate, anserine, arabitol, arabonate, arachidonate, arginine, aspartate, azelate (nonanedioate), β-alanine, betaine, butyrylcarnitine, cadaverine, caffeine, caprate, caproate, caprylate, carnitine, choline phosphate, cis-aconitate, citramalate, citrate, citrulline, creatine, creatinine, deoxycarnitine, dihomo-linoleate, docosadioate, eicosanodioate, erythritol, erythronate, ethanolamine, fructose, fucose, fumarate, galactose, gamma-aminobutyrate, gluconate, glucose, glutamate, glutamine, glutarate (pentanedioate), glycerate, glycerol 3-phosphate, glycine, glycolate (hydroxyacetate), glycylglycine, lycylisoleucine, glycylleucine, glycylphenylalanine, glycylproline, glycyltyrosine, glycylvaline, guanine, guanosine, gulono-1,4-lactone, heptaethylene glycol, heptanoate, hexaethylene glycol, hippurate, histidine, hypoxanthine, indoleacetate, inositol 1-phosphate, isoleucine, isoleucylisoleucine, isoleucylleucine, isomaltose, isovalerate, lactate, leucine, leucylisoleucine, leucylleucine, levulinate (4-oxovalerate), lysine, malate, mannose, myoinositol, N6-acetyllysine, N-acetylgalactosamine, N-acetylglucosamine, N-acetylleucine, N-acetylmethionine, N-acetylneuraminate, N-acetylornithine, N-acetylphenylalanine, N-acetylputrescine, N-acetylserine, N-carbamoylaspartate, ornithine, orotate, oxalate (ethanedioate), pantothenate, paraxanthine, p-cresol sulfate, phenylacetate, phenylalanine, phenylalanylphenylalanine, phenyllactate, phosphate, picolinate, pipecolate, proline, propionylcarnitine, putrescine, pyroglutamine, pyroglutamylglutamine, pyroglutamylglycine, pyrophosphate, pyruvate, quinate, ribose, saccharin, scylloinositol, serine, sorbitol, stachydrine, succinate, succinylcarnitine, tetraethylene glycol, theobromine, threonate, threonine, thrconylphcnylalanine, hymidinc, thymine, trihydroxybutane, tryptophan, tyrosine, undecanedioate, uracil, urate, urea, urocanate, valine, valinylglutamate, vanillate, vanillin, xanthine, xylonite, xylose, 1,5-anhydroglucitol, 10-heptadecenoate, 10-nonadecenoate, 10-undecenoate, 13-methylmyristic acid, 1-eicosadienoylglycerophosphocholine, 1-eicosatrienoylglycerophosphocholine, I-oleoylglycerol (1-monoolein), 1-palmitoylglycerophosphoethanolamine, 1-palmitoylglycerophosphoinositol, 1-palmitoylplasmenylethanolamine, 21-hydroxypregnenolone disulfate, 2-aminobutyrate, 2-linoleoylglycerophosphoethanolamine, 2-oleoylglycerophosphoethanolamine, 2-palmitoylglycerophosphocholine, 3-hydroxydecanoate, 4-hydroxyphenylpyruvate, 5alpha-androstan-3alpha, 5alpha-androstan-3β, alpha-hydroxyisocaproate, arachidonate, cis-4-decenoyl carnitine, cortisone, dihomo-linoleate, dihomo-linolenate, dimethylglycine, docosadienoate, docosahexaenoate, docosapentaenoate, eicosapentaenoate, fructose, glutamate, glycerol, glycerol 2-phosphate, hippurate, isovalerate, lactate, laurate, laurylcarnitine, linoleate, lysine, mannose, nonadecanoate, octanoylcarnitine, palmitate, palmitoleate, pregn steroid monosulfate and pyridoxate. In some embodiments, biomarkers may be selected from arginine, creatine, dihomo-linoleate, tyrosine, beta-endorphin, chromogranin A, annexin I, BDNF, 3-methyl-1-oxo-butyrate, or linolenate. In other embodiments, biomarkers may be selected from β-endorphin, chromogranin A, Annexin and BDNF.

The methods used herein include establishing reference levels for biomarkers in the bodily fluids of individuals. Such reference levels may be from a healthy population, a population suffering from the same or different sleep disorders, and/or reference levels from the individual being tested taken at an earlier time point in the treatment of the sleep disorder. These reference levels may be used as a comparison for biomarker levels in samples (such as saliva) obtained from an individual suspected of or suffering from a sleep disorder. An individual is determined to have a sleep disorder if their biomarker levels are within the reference levels of individuals with a sleep disorder or greater than reference levels of a normal, healthy population. For example, reference levels for a diagnosis of a sleep disorder from a population previously diagnosed with a sleep disorder may be as follows: between about 1491 to about 2100 pg/ml for β-endorphin, about 3.4 to about 5.0 pmol/mg protein for chromogranin A, about 56 to about 84 ng/ml for Annexin 1, about 502 to about 679 pg/ml for BDNF, about 53 to about 71.8 μmol/ml for 3-methyl-2-oxobutyrate, about 0.48 to about 0.75 μmol/ml for creatine, about 0.58 to about 0.85 μmol/ml for tyrosine, about 0.32 to about 0.65 μmol/ml for arginine, about 35 to about 43 μmol/ml for dihomo-linolenate and about 37 to about 52 pmol/ml for linolenate. Reference ranges for a normal (alertness) healthy population are as follows: between about 1200 to about 1490 pg/ml for β-endorphin, about 2.0 to about 3.3 pmol/mg protein for chromogranin A, about 8 to about 55 ng/ml for Annexin 1, about 199 to about 488 pg/ml for BDNF, about 19.2 to about 52 μmol/ml for 3-mcthyl-2-oxobutyrate, about 0.42 to about 0.47 increase in pmol/ml for creatine, about 0.33 to about 0.57 μmol/ml for tyrosine, about 0.20 to about 0.32 μmol/ml for arginine, about 18 to about 34 μmol/ml for dihomo-linolenate and about 18 to about 36 μmol/ml for linolenate. Biomarkers in an individual being tested for sleepiness may be compared to one or more of these reference ranges. Reference levels may additionally be smaller subsets of these ranges or a single point within one of these ranges.

In another embodiment, the reference levels for biomarkers are established based on biomarker levels in a sample taken from an individual at an earlier point in time. The individual is determined to be responding to treatment for the sleep disorder if the relative amounts of the biomarkers in the biological sample have altered favorably from the biomarker levels in a biological sample taken at an earlier first time point from the same individual; i.e. trend towards normal biomarker levels. Similarly, the disease state of the individual may be progressing if the biomarker levels in a biological fluid sample are increasing in comparison to biomarker levels in the individual taken at an earlier time point and/or in reference to control levels.

In some embodiments, a method of diagnosing sleepiness may include collecting a saliva sample from an individual suspected of suffering a sleep disorder, applying the saliva sample to a solid support on which agents that bind to arginine, creatine, dihomo-linoleate, tyrosine, beta-endorphin, chromogranin A, annexin I, BDNF, 3-methyl-1-oxo-butyrate, or linolenate have been affixed. In some embodiments two agents may be affixed. In other embodiments three, four, five, six, seven, eight or nine agents may be fixed. The agents bind to the respective biomarkers in the sample to produce a measurable complex of each selected biomarker. The quantitative or qualitative amount of the biomarkers creates a first individual biomarker profile. Such a first individual biomarker profile may be compared to the reference levels for specific biomarkers. The reference levels may be obtained from a healthy population, a population diagnosed with sleep disorders, or the biomarker levels for the individual at a previous point in time. One, two or more biomarker levels which exceed an arginine reference level of about 0.32 μmol/ml, a creatine reference level of about 0.47 μmol/ml, a dihomo-linoleate reference level of about 34 μmol/ml, a tyrosine reference level of about 0.57 μmol/ml, a beta-endorphin reference level of about 1490 pg/ml, a BDNF reference level of about 488 pg/ml, a 3-methyl-oxobutyrate level of about 52 μmol/ml, a linoleate reference level of about 36 μmol/ml, a chromogranin A reference level of about 3.3 μmol/mg protein, and an annexin I reference level of about 55 ng/ml may be indicative of a sleep disorder.

The saliva samples from the individual suspected of having the sleep disorder may be collected within the same or different times of day as saliva samples used to determine the reference levels. In some embodiments, the saliva samples may be collected in the same or a different manner than the saliva sample used to determine the reference level. In further embodiments, the saliva sample from the individual suspected of having the sleep disorder may be tested for levels of the biomarkers using the same or a different type of assay than the assay used to determine the reference levels of the biomarkers.

In further embodiments, the effectiveness of a treatment for a sleep disorder may be monitored by collecting a first saliva sample from an individual diagnosed with the sleep disorder, applying the first saliva sample to a first solid support on which a group of agents, which in combination bind to at least two different single biomarkers selected from arginine, creatine, dihomo-linoleate, tyrosine, beta-endorphin, chromogranin A, annexin I, BDNF, 3-methyl-1-oxo-butyrate, or linolenate have been affixed. Each agent binds to a different single biomarker to form a measurable complex of each of the different single biomarkers. The level of each biomarker in a measurable complex is assessed either qualitatively or quantitatively to form a first individual biomarker profile. The individual may then be treated for a sleep disorder before the collection of a second saliva sample during or after the course of treatment. The second saliva sample is applied to a second solid support on which the group of agents which bind to the different single biomarker on the first solid support have been affixed to form a measurable complex of each of the different single biomarkers. The amount of biomarker in the measurable complex formed by the second saliva sample is determined either qualitatively or quantitatively to create a second individual biomarker profile. The first individual biomarker profile and the second individual biomarker profile are then compared. In some embodiments, a decrease in levels of two or more of arginine, creatine, dihomo-linoleate, tyrosine, beta-endorphin, chromogranin A, annexin I, BDNF, 3-methyl-1-oxo-butyrate, or linolenate in the second individual biomarker profile relative to the first individual biomarker profile indicates that the treatment is effective. The first and second saliva samples may be taken at the same or different times of day, using the same or different collection methods and the same or different types of assays.

In some embodiments, a kit for determining whether a patient has a sleep disorder or evaluation of a treatment for a sleep disorder may include a group of test strips, each configured to produce a fluorescence level proportional to an amount present on one of the group of test strips of at least two biomarkers selected from arginine, creatine, dihomo-linoleate, tyrosine, beta-endorphin, chromogranin A, annexin I, BDNF, 3-methyl-1-oxo-butyrate, or linolenate and/or a reading device configured to read the fluorescence level proportional to the level of each biomarker on each of the group of test strips after each of the group of test strips are exposed to a saliva sample. A positive indication of a sleep disorder would be an arginine level greater than about 0.32 μmol/ml, a creatine level greater than about 0.47 μmol/ml, a dihomo-linoleate level greater than about 34 μmol/ml, a tyrosine level greater than about 0.57 μmol/ml, a beta-endorphin level greater than about 1490 pg/ml, a BDNF level greater than about 488 pg/ml, a 3-methyl-oxobutyrate level greater than about 52 μmol/ml, a linoleate level greater than about 36 μmol/ml, a chromogranin A level greater than about 3.3 μmol/mg protein, or an annexin I level greater than about 55 ng/ml. In some embodiments biomarker levels may fall within a range such as an arginine level greater than about 0.32 but less than about 0.65 μmol/ml, a creatine level greater than about 0.48 but less than about 0.75 μmol/ml, a dihomo-linoleate level greater than about 35 but less than about 43 μmol/ml, a tyrosine level greater than about 0.58 to about 0.85 μmol/ml, beta-endorphin level greater than about 1491 but less than about 2100 pg/ml, a BDNF reference level greater than about 502 but less than about 679 pg/ml, a 3-methyl-oxobutyrate level greater than about 53 but less than about 71.8 μmol/ml, a linoleate level greater than about 37 but less than about 52 μmol/ml, a chromogranin A level greater than about 3.4 but less than about 5.0 μmol/mg protein, an annexin I level greater than about 6 but less than about 84 ng/ml. In some embodiments, such a kit may further include instructions to take the saliva sample from the patient in a same manner as saliva samples used to determine the reference levels. In further embodiments, such a kit may include instructions to take the saliva sample from the patient within a same time of day window as saliva samples used to determine the reference levels. In additional embodiments, such a kit may include instructions to take the saliva sample from the patient in a same manner and within a same time of day window as saliva samples used to determine the reference levels.

These and other embodiments, features and potential advantages will become apparent with reference to the following description.

### DETAILED DESCRIPTION

### Glossary

“Biomarker” in this context refers to a characteristic that is objectively measured and evaluated as an indicator of normal biological processes, pathogenic processes, or pharmacologic responses to a therapeutic intervention.

“Biomarker panel” in this context refers to a set of biomarkers that can be used alone, together, or in sub-combinations for the detection, diagnosis, prognosis, staging, or monitoring of a disease or condition, based on detection values for the set of biomarkers. The biomarkers within the panel of biomarkers used herein include, but are not limited to, 1,2-propanediol, 1,5-anhydroglucitol, 2,3-dihydroxyisovalerate, 2′-deoxyguanosine, 2′-deoxyinosine, 2-hydroxy-3-methylvalerate, 2-hydroxybutyrate, 2-hydroxyglutarate, 3-(4-hydroxyphenyl)lactate, 3-(4-hydroxyphenyl)propionate, 3-dehydrocarnitine, 3-hydroxybutyrate, 3-methyl-2-oxobutyrate, 3-methyl-2-oxovalerate, 3-phenylpropionate, 4-hydroxybutyrate, 4-hydroxyphenylacetate, 4-methyl-2-oxopentanoate, 5,6-dihydrothymine, 5-aminovalerate, 5-oxoproline, 8-hydroxyoctanoate, adenine, agmatine, alanine, alanylalanine, alanylisoleucine, alanylleucine, alanylproline, alanylvaline, allantoin, alpha-glutamylglutamate, alpha-hydroxyisocaproate, alpha-hydroxyisovalerate, alpha-ketoglutarate, anserine, arabitol, arabonate, arachidonate, arginine, aspartate, azelate (nonanedioatc), β-alanine, betainc, butyrylcarnitine, cadaverine, caffeine, caprate, caproate, caprylate, carnitine, choline phosphate, cis-aconitate, citramalate, citrate, citrulline, creatine, creatinine, deoxycarnitine, dihomo-linoleate, docosadioate, eicosanodioate, erythritol, erythronate, ethanolamine, fructose, fucose, furnarate, galactose, gamma-aminobutyrate, gluconate, glucose, glutamate, glutamine, glutarate (pentanedioate), glycerate, glycerol 3-phosphate, glycine, glycolate (hydroxyacetate), glycylglycine, lycylisoleucine, glycylleucine, glycylphenylalanine, glycylproline, glycyltyrosine, glycylvaline, guanine, guanosine, gulono-1,4-lactone, heptaethylene glycol, heptanoate, hexaethylene glycol, hippurate, histidine, hypoxanthine, indoleacetate, inositol 1-phosphate, isoleucine, isoleucylisoleucine, isoleucylleucine, isomaltose, isovalerate, lactate, leucine, leucylisoleucine, leucylleucine, levulinate (4-oxovalerate), lysine, malate, mannose, myoinositol, N6-acetyllysine, N-acetylgalactosamine, N-acetylglucosamine, N-acetylleucine, N-acetylmethionine, N-acetylneuraminate, N-acetylornithine, N-acetylphenylalanine, N-acetylputrescine, N-acetylserine, N-carbamoylaspartate, ornithine, orotate, oxalate (ethanedioate), pantothenate, paraxanthine, p-cresol sulfate, phenylacetate, phenylalanine, phenylalanylphenylalanine, phenyllactate, phosphate, picolinate, pipecolate, proline, propionylcarnitine, putrescine, pyroglutamine, pyroglutamylglutamine, pyroglutamylglycine, pyrophosphate, pyruvate, quinate, ribose, saccharin, scylloinositol, serine, sorbitol, stachydrine, succinate, succinylcarnitine, tetraethylene glycol, theobromine, threonate, threonine, threonylphenylalanine, hymidine, thymine, trihydroxybutane, tryptophan, tyrosine, undecanedioate, uracil, urate, urea, urocanate, valine, valinylglutamate, vanillate, vanillin, xanthine, xylonite, xylose, 1,5-anhydroglucitol, 10-heptadecenoate, 10-nonadecenoate, 10-undecenoate, 13-methylmyristic acid, 1-eicosadienoylglycerophosphocholine, 1-eicosatrienoylglycerophosphocholine, 1-oleoylglycerol (1-monoolein), 1-palmitoylglycerophosphoethanolamine, 1-palmitoylglycerophosphoinositol, 1-palmitoylplasmenylethanolamine, 21-hydroxypregnenolone disulfate, 2-aminobutyrate, 2-linoleoylglycerophosphoethanolamine, 2-oleoylglycerophosphoethanolamine, 2-palmitoylglycerophosphocholine, 3-hydroxydecanoate, 4-hydroxyphenylpyruvate, 5alpha-androstan-3alpha, 5alpha-androstan-3 i, alpha-hydroxyisocaproate, arachidonate, cis-4-decenoyl carnitine, cortisone, dihomo-linoleate, dimethylglycine, docosadienoate, docosahexaenoate, docosapentaenoate, eicosapentaenoate, fructose, glutamate, glycerol, glycerol 2-phosphate, Hippurate, isovalerate, lactate, laurate, laurylcarnitine, linoleate, lysine, mannose, nonadecanoate, octanoylcarnitine, palmitate, palmitoleate, pregn steroid monosulfate and pyridoxate.

“Concentration” or “level” as used herein can refer to an absolute or relative quantity.

“Measuring,” “detecting,” or “taking a measurement” as used in this context refers to a quantitative or qualitative determination of the amount or concentration of the biomarker in a particular sample.

“Reference value” in this context refers to an absolute value, a relative value, a value that has an upper and/or lower limit, a range of values, an average value, a median value, a mean value, a shrunken centroid value, a value as compared to a particular control or baseline value or a combination thereof. It is to be understood that other statistical variables may be used in determining the reference value.

“REM sleep” in this context refers to rapid eye movement sleep.

“Sleepiness” in this context refers to a condition caused by sleep deprivation, disease, coma, sleep fragmentation and disorders or conditions generally known in the art or otherwise described herein.

“Therapeutic effect”, “therapeutic activity” or “therapeutic action” in this context refers to a desired pharmacological activity of the agent.

“Treatment” in this context refers to refers to the alleviation, amelioration, and/or stabilization of symptoms, as well as a delay in the progression of symptoms of a particular disorder through the use of some external drug, device or technology.

### Description

Provided herein is a non-invasive means for diagnosing, detecting, and monitoring sleepiness including, REM and NREM sleep loss, using a specific subset of one or more biomarkers. As disclosed herein, multiple biomarkers in the bodily fluids of a mammalian individual may be quantitatively or qualitatively measured alone or in combination as a diagnostic for sleepiness, including early diagnosis of sleep disorders, as well as a determiner of risk of developing sleepiness due to sleep disorders. Levels of biomarkers may additionally be used to monitor the progression and severity of sleepiness caused by sleep disorders, and determine the effectiveness of a particular treatment in addressing sleepiness caused by a sleep disorder.

Sleepiness which may be detected, diagnosed, predicted and/or treated using one or more of the methods described herein may be caused by any of a number of environmental and/or physiological causes. For example, such sleepiness may be caused by one or more sleep disorders including, but not limited to, dyssomnias, parasomnias, sleep disorders associated with medical/psychiatric disorders, and proposed sleep disorders. Dyssomnias are typically divided into intrinsic sleep disorders, extrinsic sleep disorders, and circadian rhythm sleep disorders. Intrinsic sleep disorders include psycho-physiological insomnia, sleep state misperception, idiopathic insomnia, narcolepsy, recurrent hypersomnia, idiopathic hypersomnia, post-traumatic hypersomnia, obstructive sleep apnea syndrome, central sleep apnea syndrome, central alveolar hypoventilation syndrome, periodic limb movement disorder, rhythmic movement disorder and restless legs syndrome. Extrinsic sleep disorders include inadequate sleep hygiene, environmental sleep disorder, altitude insomnia, adjustment sleep disorder, insufficient sleep syndrome, limit-setting sleep disorder, sleep-onset association disorder, food allergy insomnia, nocturnal eating (drinking) syndrome, hypnotic-dependent sleep disorder, stimulant-dependent sleep disorder, alcohol-dependent sleep disorder, and toxin-induced sleep disorder. Circadian rhythm sleep disorders include time zone (jet lag) syndrome, space environments such as no day and night difference, differences in times between living on Earth and working on a Mars Mission, shift work sleep disorder, irregular sleep-wake pattern, delayed sleep phase syndrome, advanced sleep phase syndrome, and non-24-hour sleep-wake disorder. Proposed sleep disorders include short sleeper, someone who regularly sleeps less than 75 percent of the sleep time usually required in his or her age group, and feels no negative effects from this shortened sleep; long sleepers who need substantially more sleep than most people, usually means sleeping 10 hours or more for adults with normal sleep timing and structure; subwakefulness syndrome, in which individuals complain about a lack of daytime alertness, but have no nighttime sleep disruption and seem to be getting adequate sleep; fragmentary myoclonus which manifests by brief, involuntary jerks or twitches; sleep hyperhydrosis (night sweats), individuals who sweat heavily at night without any signs of fever or other disorders; menstrual-associated sleep disorder including premenstrual insomnia, premenstrual hypersomnia, and menopausal insomnia; pregnancy-associated sleep disorder such as insomnia or excessive sleep and sleepiness; terrifying hypnagogic hallucinations, in which common, unthreatening hypnagogic images can turn threatening, and seem real, partly as a result of how quickly they follow wakefulness; sleep-related laryngospasm, in which a spasm of the throat closes off the airway and halts breathing during sleep even though the individuals do not have apnea, with episodes lasting anywhere from a few seconds to as long as five minutes; sleep choking syndrome in which individuals have episodes of choking almost nightly, and sometimes more than once a night though the patients don't suffer from obstructive sleep apnea, nightmares, night terrors, or other forms of nocturnal anxiety attacks.

The methods described herein may additionally be used to detect or treat a subject suffering from sleepiness due to parasomnia. Parasomnias are sleep disorders that involve abnormal movements, behaviors, emotions, perceptions, and dreams that occur while falling asleep, during sleep, between sleep stages, or during arousal from sleep. Arousal disorders include confusional arousals, sleepwalking, sleep talking, and sleep (or night) terrors. It is mainly associated with REM sleep and may include nightmares, sleep paralysis, impaired sleep-related penile erections, sleep-related painful erections, REM sleep-related sinus arrest, and REM sleep behavior disorder. Other parasomnias may include sleep bruxism, sleep enuresis, sleep-related abnormal swallowing syndrome, nocturnal paroxysmal dystonia, sudden unexplained nocturnal death syndrome, primary snoring, infant sleep apnea, congenital central hypoventilation syndrome, sudden infant death syndrome, and benign neonatal sleep myoclonus. The method may also be used to diagnose and treat a subject having a sleep disorder associated with a medical or psychiatric condition. Sleep disorders associated with neurological disorders may include cerebral degenerative disorders, dementia, Parkinsonism, fatal familial insomnia, sleep-related epilepsy, electrical status epilepticus of sleep, and sleep-related headaches. Sleep disorders associated with other medical disorders may include sleeping sickness, nocturnal cardiac ischemia, chronic obstructive pulmonary disease, sleep-related asthma, sleep-related gastroesophageal reflux, peptic ulcer disease, and fibrositis syndrome.

Additional causes of sleepiness which may be detected and/or treated according to the methods provided herein include, but are not limited to, short sleeper, long sleeper, subwakefulness syndrome, fragmentary myoclonus, sleep hyperhidrosis, menstrual-associated sleep disorder, pregnancy-associated sleep disorder, terrifying hypnogogic hallucinations, sleep-related neurogenic tachypnea, sleep-related larnyngospasm, and sleep choking syndrome. Other sleep disorders are associated with sky divers, water divers, long-haul drivers, young people especially males under age of 28, parents of young children, police officers, pilots, health professionals, i.e., physicians, military professionals, individuals being treated with certain sedating medications, i.e., anti-depressants, and individuals who consume alcohol or certain drugs such as those classified by the U.S. National Institute for Drug Abuse (NIDA) as drugs of abuse including marijuana, heroin, amphetamines, phencyclidine and cocaine.

Biomarkers may be measured individually or as part of a panel of biomarkers. In some embodiments, biomarkers for sleepiness are attached to a surface such that levels might be obtained directly or indirectly. In further embodiments, sleepiness biomarker-specific affinity reagents are bound to a solid support to provide separation of the sleepiness biomarkers in biological samples, particularly saliva. Sleepiness biomarker complex formation leads to at least one sleepiness diagnostic biomarker bound to a reagent specific for the biomarker, wherein said biomarker is attached to a surface. Binding or complex formation can be measured qualitatively or quantitatively. Both standard and competitive formats for these assays including point of care systems are known in the art. The bound sleepiness biomarkers are detected using a mixture of appropriate detection reagents, including fluorescent dye-based or other visual systems that specifically bind various sleepiness biomarkers.

The compositions and methods described can be used in methods to screen subjects that have or are at risk for developing sleepiness from sleep disorders; to monitor individuals who are undergoing therapies for sleep disorders; or to select or modify therapies or interventions for use in treating subjects with a sleep disorder. Further described are compositions and methods for laboratory and point-of-care tests for measuring biomarkers in a sample from an individual.

In various embodiments described herein are methods for predicting the development of sleepiness in an individual, determining the prognosis of sleepiness in an individual, diagnosing a sleep disorder in an individual, or treating a sleep disorder in an individual by measuring the levels of biomarkers in an individual being monitored using a single biomarker or a biomarker panel and comparing those levels to a reference range and/or level in a normal control, a population with a sleep disorder, a statistically significant number of samples drawn from a population that includes the individual, and/or levels taken at a previous time point from the individual being monitored. In one aspect, provided herein is a method of diagnosing an individual suffering from sleepiness comprising taking a biological sample from the individual, measuring the levels of biomarkers in a biomarker panel and correlating the measurement with the disease. In these embodiments, to make comparisons to the subject-derived sample, the amounts of reference biomarkers are similarly calculated. Subjects identified as having sleep disorders, or at increased risk of developing sleep disorders may receive therapeutic and/or prophylactic treatment for the sleep disorder based on biomarker levels or other diagnostic factors. A sleep disorder is considered to be progressive if the amount of biomarker for sleepiness moves further away from the reference range and/or level of a healthy population over time, whereas a disease is not progressive if the amount of biomarkers remains constant over time, approaches the reference level of a healthy individual, and/or the symptoms of the sleep disorder stabilize or improve.

The use of biomarkers allows for real-time testing to predict, diagnose, and monitor disease and treatment of sleep disorders. Measurement of any combination of biomarkers described herein may be used to assemble a biomarker panel. The combination may refer to the measurement of an entire set or any subset or sub-combination of biomarkers thereof. Specifically, the detection of a plurality of biomarkers in a sample can increase the sensitivity and/or specificity of the test. Thus in various embodiments, a biomarker panel as described herein may be used to measure 1, 2, 3, 4, 5, 6, 7, 8, 9, 10, 11, 12, 13, 14, 15, 16, 17, 18 or more biomarkers. In exemplary embodiments, a biomarker panel may measure, 1, 2, 3, 4, 5, 6, 7, 8, 9 or 10 biomarkers. Relevant biomarkers for predicting, diagnosing and monitoring of sleep disorders include, but are not limited to, 1,2-propancdiol, 1,5-anhydroglucitol, 2,3-dihydroxyisovalerate, 2′-deoxyguanosine, 2′-deoxyinosine, 2-hydroxy-3-methylvalerate, 2-hydroxybutyrate, 2-hydroxyglutarate, 3-(4-hydroxyphenyl)lactate, 3-(4-hydroxyphenyl)propionate, 3-dehydrocamitine, 3-hydroxybutyrate, 3-methyl-2-oxobutyrate, 3-methyl-2-oxovalerate, 3-phenylpropionate, 4-hydroxybutyrate, 4-hydroxyphenylacetate, 4-methyl-2-oxopentanoate, 5,6-dihydrothymine, 5-aniinovalerate, 5-oxoproline, 8-hydroxyoctanoate, adenine, agmatine, alanine, alanylalanine, alanylisoleucine, alanylleucine, alanylproline, alanylvaline, allantoin, alpha-glutamylglutamate, alpha-hydroxyisocaproate, alpha-hydroxyisovalerate, alpha-ketoglutarate, anserine, arabitol, arabonate, arachidonate, arginine, aspartate, azelate (nonanedioate), β-alanine, βine, butyrylcarnitine, cadaverine, caffeine, caprate, caproate, caprylate, carnitine, choline phosphate, cis-aconitate, citramalate, citrate, citrulline, creatine, creatinine, deoxycarnitine, dihomo-linoleate, docosadioate, eicosanodioate, erythritol, erythronate, ethanolamine, fructose, fucose, fumarate, galactose, gamma-aminobutyrate, gluconate, glucose, glutamate, glutamine, glutarate (pentanedioate), glycerate, glycerol 3-phosphate, glycine, glycolate (hydroxyacetate), glycylglycine, lycylisoleucine, glycyllcucine, glycylphenylalanine, glycylproline, glycyltyrosine, glycylvaline, guanine, guanosine, gulono-1,4-lactone, heptaethylene glycol, heptanoate, hexaethylene glycol, hippurate, histidine, hypoxanthine, indoleacetate, inositol 1-phosphate, isoleucine, isoleucylisoleucine, isoleucylleucine, isomaltose, isovalerate, lactate, leucine, leucylisoleucine, leucylleucine, levulinate (4-oxovalerate), lysine, malate, mannose, myoinositol, N6-acetyllysine, N-acetylgalactosamine, N-acetylglucosamine, N-acetylleucine, N-acetylmethionine, N-acetylneuraminate, N-acetylornithine, N-acetylphenylalanine, N-acetylputrescine, N-acetylserine, N-carbamoylaspartate, ornithine, orotate, oxalate (ethanedioate), pantothenate, paraxanthine, p-cresol sulfate, phenylacetate, phenylalanine, phenylalanylphenylalanine, phenyllactate, phosphate, picolinate, pipecolate, proline, propionylcarnitine, putrescine, pyroglutamine, pyroglutamylglutamine, pyroglutamylglycine, pyrophosphate, pyruvate, quinate, ribose, saccharin, scylloinositol, serine, sorbitol, stachydrine, succinate, succinylcarnitine, tetraethylene glycol, theobromine, threonate, threonine, threonylphenylalanine, hymidine, thymine, trihydroxybutane, tryptophan, tyrosine, undecanedioate, uracil, urate, urea, urocanate, valine, valinylglutamate, vanillate, vanillin, xanthine, xylonite, xylose, 1,5-anhydroglucitol, 10-heptadecenoate, 10-nonadecenoate, 10-undecenoate, 13-methylmyristic acid, 1-eicosadienoylglycerophosphocholine, 1-eicosatrienoylglycerophosphocholine, 1-oleoylglycerol (1-monoolein), 1-palmitoylglycerophosphoethanolamine, 1-palmitoylglycerophosphoinositol, 1-palmitoylplasmenylethanolamine, 21-hydroxypregnenolone disulfate, 2-aminobutyrate, 2-linoleoylglycerophosphoethanolamine, 2-oleoylglycerophosphoethanolamine, 2-palmitoylglycerophosphocholine, 3-hydroxydecanoate, 4-hydroxyphenylpyruvate, 5alpha-androstan-3alpha, 5alpha-androstan-313, alpha-hydroxyisocaproate, arachidonate, cis-4-decenoyl carnitine, cortisone, dihomo-linoleate, dihomo-linolenate, dimethylglycine, docosadienoate, docosahexaenoate, docosapentaenoate, eicosapentaenoate, fructose, glutamate, glycerol, glycerol 2-phosphate, hippurate, isovalerate, lactate, laurate, laurylcarnitine, linoleate, lysine, mannose, nonadecanoate, octanoylcarnitine, palmitate, palmitoleate, pregn steroid monosulfate and pyridoxate. In some embodiments, biomarkers may be one or more of salivary β-endorphin, chromogranin A, Annexin 1, neurotrophin brain derived neurotrophic factor (BDNF), 3-methyl-2-oxobutyrate, creatine, tyrosine, arginine, dihomo-linolenate and linolenate. In some embodiments a single biomarker may be measured. In other embodiments a biomarker panel may be used to measure one or more of the listed biomarkers in any combination. In various embodiments, diagnostic tests that use these biomarkers alone or in combination show a sensitivity of at least about 60%, about 61%, about 62%, about 70%, about 75%, about 80%, about 83%, about 85%, about 86%, about 88%, about 90%, about 95% as shown in Table 2. Such diagnostic tests may additionally have a specificity of at least about 60%, about 61%, about 62%, about 75%, about 70%, about 75%, about 80%, about 83%, about 85%, about 86%, about 88%, about 90% or more. Biomarkers as used herein may be used alone or in combination with other diagnostic tools such as, but not limited to, an electroencephalogram (EEG), electrooculogram (EOG), electromyogram (EMG) or electrocardiogram (ECG). Individuals may additionally be evaluated using the results from one or more sleep questionnaires including, but not limited to a general medical questionnaire, Quality of Life questionnaire, Pittsburgh sleep quality index, Epworth sleepiness scale, IQ questionnaire, Hamilton depression rating scale, Atypical depression supplement or insomnia severity index questionnaire (Zavada A, Gordijn M C, Beersma D G, Daan S, Roenneberg T, Comparison of the Munich Chronotype Questionnaire with the Horne-Ostberg's Morningness-Eveningness Score. Chronobiol Int. 2005; 22(2):267-78; Buysse D J, Reynolds C F 3rd, Monk T H, Berman S R, Kupfer D J. The Pittsburgh Sleep Quality Index: a new instrument for psychiatric practice and research. Psychiatry Res. 1989 May; 28(2):193-213; Johns M W. A new method for measuring daytime sleepiness: The Epworth Sleepiness Scale. Sleep. 1991; 14: 540-545; Schwab J J, Bialow M, Brown J M, Holzer C E. Diagnosing depression in medical inpatients. Ann Intern Med. 1967 October; 67(4):695-707; Bastien C H, Vallieres A, Morin C M. Validation of the Insomnia Severity Index as an outcome measure for insomnia research. Sleep Med. 2001 July; 2(4):297-307; Viola A U I, Tobaldini E, Chellappa S L, Casali K R, Porta A, Montano N. Short-term complexity of cardiac autonomic control during sleep: REM as a potential risk factor for cardiovascular system in aging. PLoS One. 2011 Apr. 22; 6(4):e19002).

Biomarkers as described herein may be used to identify individuals destined to become affected or who are in the “preclinical” stages of a sleep disorder; identify a reduction in disease heterogeneity in individuals participating in clinical trials or epidemiologic studies; reflect the natural history of a disease encompassing the phases of induction, latency and detection; and evaluate potential targets for treatment of a sleep disorder. A biomarker as identified herein may be suitable for the early diagnosis of a disease, either as part of a routine screening exam or at the first sign of a symptom. Biomarkers may also appear or disappear over the course of disease progression and thus be useful in determining the prognosis of a disease within an individual. Levels of the biomarker(s) may change as a drug therapy is started, adjusted or discontinued, ultimately aiding in the monitoring of the patient's response to that particular therapy.

A good biomarker should be accurate and reliable, with demonstrable safety and effectiveness for intended use. (In vitro Diagnostics-Role in Efficacy Biomarker Assessment Minimal Residual Disease (MRD) as a Surrogate Endpoint in Acute Lymphoblastic Leukemia (ALL) Workshop Silver Spring, Md. April, 2012 Elizabeth Mansfield, PhD FDA/CDRH/OIVD). The National Institute of Health's Biological Definitions Working Group indicated that a biomarker should identify a disease or abnormal condition; stage a disease or classify the extent of disease; indicate disease prognosis; and monitor clinical response to an intervention. Additionally, sets of biomarkers may be used to increase the sensitivity of tests to predict, monitor and diagnose sleepiness and underlying sleep disorders. Sets may comprise one, two, three, four, five, six, seven, eight, nine, ten or more biomarkers. These sets of biomarkers are useful for a number of purposes, for example, determining the risk of developing sleepiness, assessing the severity of the disease, monitoring sleepiness post-diagnosis, monitoring the effectiveness of therapeutic treatments and others. In some embodiments, ratios of biomarkers to each other may be useful in predicting, diagnosing and monitoring sleepiness.

Biomarkers may be collected using any means generally used from any bodily fluid including, but not limited to, saliva, blood, gingival crevicular fluid, serum, plasma, urine, nasal swab, cerebrospinal fluid, pleural fluid, synovial fluid, peritoneal fluid, amniotic fluid, gastric fluid, lymph fluid, interstitial fluid, tissue homogenate, cell extracts, saliva, sputum, stool, physiological secretions, tears, mucus, sweat, milk, semen, seminal fluid, vaginal secretions, fluid from ulcers and other surface eruptions, blisters, abscesses, and extracts of tissues including biopsies of normal and suspect tissues or any other constituents of the body which may contain the target molecule of interest. As described herein, assessment of results may be qualitative or quantitative depending upon the specific method of detection employed.

While biomarkers may be identified from any bodily fluid, human saliva collection is less invasive than that of blood for serum/plasma analyses and many if not all, blood components are reflected in saliva. Saliva is the product of three pairs of major salivary glands, parotid, submandibular and sublingual, and multiple minor salivary glands lying beneath the oral mucosa.

Saliva is an attractive diagnostic fluid because salivary testing is low cost, non-invasive, and easy to collect and process. For example, saliva may be collected by stimulated or unstimulated means as described in Example I, below. In some embodiments, the saliva collecting device may comprise an absorbent pad material that is made up of hydrophilic materials including, but not limited to, Ahlstrom materials catalog numbers 270 and 320, Schleicher & Schuell catalog numbers 300 and 900 and Filtrona Fibertec Transorb Wicks, among others. Those skilled in the art know that absorbent pad materials may also include hydrophilic or hydrophobic components bound, or integrated into the material, such components being capable of modifying the absorption and release characteristics of the absorbent pad as well as the speed of uptake of the sample fluid under consideration. In some embodiments, the saliva collection agent may be made out of a mixture of polyethylene and polypropylene such as those described in U.S. Pat. No. 7,618,591 and U.S. Pat. No. 8,025,851.

Additional saliva collection devices which may be used for stimulated or unstimulated saliva collection include, but are not limited to, the UltraSal-2™ (Oasis Diagnostics®) saliva collection device which automatically splits the saliva specimen into two aliquots in separate collection tubes. One tube can be used for testing while the second may be used for confirmation of results or for future use. The specimen is collected by holding the mouth piece between the lips and expectorating the oral fluid through the hole in the mouth piece into the collection tubes. The process is stopped when there is sufficient specimen collected. The collection tubes are capped and the mouthpiece is discarded. The volume collected is up to the capacity of the tube; the volume recovered is 100% of the collected volume. The Versi-SAL® (Oasis Diagnostics®) as described in U.S. Pat. No. 7,618,591, is a fluid collection device that incorporates a proprietary interchangeable absorbent pad. It works by placing the device pad under the tongue and collecting saliva until a sample sufficiency indicator is triggered, usually after 1-2 minutes. The collector is then pushed down into a supplied compression tube until the pad is significantly compressed to release the absorbed saliva. The saliva specimen is forced through an outlet into a graduated tube. Recovery efficiency is about 60%. A similar procedure is used in the Super.SAL™ Universal Saliva Collection device (U.S. Pat. No. 8,025,851), a tool that uses a cylindrical absorbent pad to collect approximately 1.0-1.2 mL of saliva. Saliva is recovered with an efficiency of about 75% by means of compression of the absorbent pad through a compression tube. In the Salivette® (Sarstedt) device, saliva collection is carried out by chewing a cotton wool swab. Recovery of the saliva sample is achieved by returning the swab to the Salivette® tube and centrifuging the container. The volume collected is 1.7 ml; the volume recovered is 1.4 ml (82%). In the Intercept (OraSure Technologies) device, a pad is swabbed in the mouth for 2 to 5 minutes, the pad is inserted into a vial and snapped off at a scoring, and the vial is capped and sealed. Others devices mentioned in the literature, including: OraSure (OraSure Technologies), Saliva Sampler (Stat-Sure Diagnostics) and SalivaBio Oral Swab (Salimetrics).

The VerOFy® (Oasis Diagnostics), as described in U.S. Pat. Nos. 7,618,591 and 7,927,548, both of which are incorporated by reference herein in their entireties, incorporates rapid and standardized saliva collection with immunochromatographic test strips providing a system for delivery of immediate results in the field or at point-of-care locations. The various biomarkers are conjugated to a Europium bead that emits fluorescence that is proportional to each of the biomarkers present in the saliva sample.

The biomarkers in a bodily fluid which are used herein to predict, diagnose, or monitor sleepiness can generally be measured and detected through a variety of assays, methods and detection systems known to those of skill in the art including, but not limited to, enzyme linked immunosorbent assay (ELISA), fluorescence polarization immunoassay (FPIA) and homogeneous immunoassays, point of care tests using conventional lateral flow immunochromatography (LFA), quantitative point of care tests using determination of chemiluminescence, fluorescence, radioimmunoassays, sandwich-format assays, techniques using microfluidic or MEMS technologies, and magnetic particles, as well as latex agglutination, biosensors, gel electrophoresis, mass spectrometry (MS), gas chromatography-mass spectrometry (GC-MS), and nanotechnology based methods, re-engineering technologies (e.g. instruments utilizing sensors for biomarkers used for telemedicine purposes), epitope-based technologies, other fluorescence technologies, microarrays, lab-on-a-chip, and rapid point-of-care screening by way of example. This technology includes qualitative or quantitative measurement of levels of sleepiness and/or sleep disorder biomarkers in a biological sample such as saliva. For example, as shown in Example I, below, biomarkers may be identified using an ELISA test specific for the biomarker(s) of interest which generates a color change that can be measured using a spectrophotometer.

In some embodiments, devices used to measure one or more biomarkers may be pre-treated with magnetic particles, nanoparticles or one of a series of other molecules coated with a substance or molecule capable of specifically or non-specifically binding to components in the saliva, thereby improving the performance of the assay by blocking or obstructing interfering substances.

As seen in E. A. Shirtcliff et al., Psychoneuroendocrinology 26 (2001) 165-173, “[i]mmunoassay results for salivary testosterone, DHEA, progesterone, and estradiol are higher, whereas sIgA results are significantly lower, when samples are collected using cotton absorbent materials compared to samples collected without cotton.” “In samples collected using the polypropylene Salivette cortisol, cortisol AOPP concentrations were lower (by 60%) in comparison to measurements in whole unstimulated saliva.” (Kamodyova N, Celec P., Salivary markers of oxidative stress and Salivette collection systems. Clin Chem Lab Med. 2011 November; 49(11):1887-90. doi: 10.1515/CCLM.2011.677. Epub 2011 Aug. 23.) Additionally, “the cotton roll collection method affects the results of total protein, s-IgA, amylase and cortisol” (T.-L. Li and M. Gleeson, The cotton swab method for human saliva collection: effect on measurements of saliva flow rate and concentrations of protein, secretory immunoglobulin A, amylase and cortisol, University College London (2003) J Physiol 547P, PC5). Therefore, in some embodiments, initial, control, repeat and reference samples may be collected using the same or the same type of saliva collection method. In other embodiments, they may be collected using different collection methods.

Saliva samples may be collected using the same, different, or similar types of collection methods to those used for prior collections and/or in comparison to collection methods used for reference ranges or a specific reference level. As different types of collection methods may yield different biomarker levels, in some embodiments, all saliva samples may be collected in the same manner. For example, the original sample and repeat samples may be collected using the same type of collection method used in determining the reference ranges and/or levels. In some embodiments, salivary samples are taken multiple times a day such as early in the morning, afternoon and evening or a subset thereof. In additional embodiments, salivary samples can be taken at specific times once or at multiple occasions during the day.

Along with collection methods, biomarker levels can additionally be affected by a number of other factors including, but not limited to, gender, saliva flow rates, collection location, oral inflammatory disease, smoking, screening questions, circadian rhythms, blood and environmental contamination, certain medications, and stability during storage. (Shirtcliff E A, Granger D A, Schwartz E, Curran M J. Use of salivary biomarkers in biobehavioral research: cotton-based sample collection methods can interfere with salivary immunoassay results. Psychoneuroendocrinology. 2001 February; 26(2):165-73; Tomoaki Kozaki, Soomin Lee, Takayuki Nishimura, Tetsuo Katsuura, Akira Yasukouchi, Effects of saliva collection using cotton swabs on melatonin enzyme immunoassay Journal of Circadian Rhythms 2011, 9:1; Roslinda Mohamed, Jennifer-Leigh Campbell, Justin Cooper-White, Goce Dimeski, and Chamindie Punyadeera, The impact of saliva collection and processing methods on CRP, IgE, and Myoglobin immunoassays, Mohamed et al. Clinical and Translational Medicine 2012, 1:19; T.-L. Li and M. Gleeson, The cotton swab method for human saliva collection: effect on measurements of saliva flow rate and concentrations of protein, secretory immunoglobulin A, amylase and cortisol, University College London (2003) J Physiol 547P, PC5; Ohshiro K, Rosenthal D I, Koomen J M, Streckfus C F, Chambers M, Kobayashi R, EI-Naggar A K. Pre-analytic saliva processing affect proteomic results and biomarker screening of head and neck squamous carcinoma. Int J Oncol. 2007 30 (3):743-9; Wiviott S D, Cannon C P, Morrow D A, Murphy S A, Gibson C M, McCabe C H, Sabatine M S, Rifai N, Giugliano R P, DiBattiste P M, Demopoulos L A, Antman E M, Braunwald E. Differential expression of cardiac biomarkers by gender in patients with unstable angina/non-ST-elevation myocardial infarction: a TACTICS-TIMI 18 (Treat Angina with Aggrastat and determine Cost of Therapy with an Invasive or Conservative Strategy-Thrombolysis In Myocardial Infarction 18) substudy. Circulation 2004; 109:580-586; Keller M, et al. (2009) A circadian clock in macrophages controls inflammatory immune responses. Proc Natl Acad Sci USA 106:21407-21412; David Soo-Quee Koh, Gerald Choon-Huat Koh, The use of salivary biomarkers in occupational and environmental medicine, Occup Environ Med. March 2007; 64(3): 202-210). Therefore, in some embodiments, results of the sample analysis from an individual are compared with a reference collected using the same method. In some embodiments, reference ranges and/or levels as used herein may be from an age-matched population, a control population, a non-sleepy control population, a healthy age-matched control population, a gender-matched control population, a sample time matched population or a combination thereof. In additional embodiments, sample analysis may be compared to reference levels taken at the same time of day. In further embodiments, sample analysis may be compared to reference levels of individuals with the same or similar genetic profiles. In additional embodiments, sample analysis may compare levels in the same individual at different time points during their treatment.

The methods used herein include establishing reference ranges and/or levels for biomarkers. The reference ranges and/or levels may be established from individuals who do not suffer from sleepiness, individuals who suffer from the same condition as the test subject or from the individual of interest at a different point in time. These reference ranges and/or levels may be used as a comparison for biomarker levels in samples (such as saliva) obtained from an individual at risk for a sleep disorder, suspected of having a sleep disorder, or under treatment for a sleep disorder. In some examples, the control is a sample from one or more subjects not known to have suffered from sleepiness. The individual is determined to be at risk for or suffer from sleepiness if the biomarker levels are statistically different in relative amounts to the biomarkers in the biological sample of a healthy control. The individual is determined to be at risk for or suffer from sleepiness if the biomarker levels are statistically equivalent to the biomarker levels in a population previously diagnosed with sleepiness and/or a sleep disorder. The individual is determined to be responding to treatment for sleepiness and/or the sleep disorder if the relative amounts of the biomarkers in the biological sample have altered from the biomarkers in a biological sample taken at an earlier first time point from the individual. The disease state of the individual may be progressing if the biomarker levels in a biological fluid decrease relative to the levels in the same individual taken at an earlier time point.

The methods used herein include establishing reference levels for biomarkers in the bodily fluids including saliva of individuals. Such reference levels may be from a sample drawn from a population of individuals who are (active) normal healthy, a population of individuals who have been diagnosed with sleepiness, or a previous sample from the individual currently being tested. For example, reference ranges for a normal (alertness) healthy population are as follows: between about 1200 to about 1490 pg/ml for β-endorphin, about 2.0 to about 3.3 pmol/mg protein for chromogranin A, about 8 to about 55 ng/ml for Annexin 1, about 199 to about 488 pg/ml for BDNF, about 19.2 to about 52 μmol/ml for 3-methyl-2-oxobutyrate, about 0.42 to about 0.47 increase in pmol/ml for creatine, about 0.33 to about 0.57 μmol/ml for tyrosine, about 0.20 to about 0.32 μmol/ml for arginine, about 18 to about 34 μmol/ml for dihomo-linolenate and about 18 to about 36 μmol/ml for linolenate.

Reference levels for a diagnosis of sleepiness generated from a population previously diagnosed with sleepiness may be as follows: between about 1491 to about 2100 pg/ml for β-endorphin, about 3.4 to about 5.0 μmol/mg protein for chromogranin A, about 56 to about 84 ng/ml for Annexin 1, about 502 to about 679 pg/ml for BDNF, about 53 to about 71.8 μmol/ml for 3-methyl-2-oxobutyrate, about 0.48 to about 0.75 μmol/ml for creatine, about 0.58 to about 0.85 μmol/ml for tyrosine, about 0.32 to about 0.65 μmol/ml for arginine, about 35 to about 43 μmol/ml for dihomo-linolenate and about 37 to about 52 μmol/ml for linolenate. Biomarkers in an individual being tested for sleepiness may be compared to one or more of these reference ranges.

In another embodiment, the reference levels for biomarkers are established based on biomarker levels in a sample taken from an individual at an earlier point in time. The individual is determined to be responding to treatment for a sleep disorder if the relative amounts of the biomarkers in the biological sample have altered favorably from the biomarker levels in a biological sample taken at an earlier first time point from the same individual; i.e. trending towards normal biomarker levels.

In some embodiments, the measured value of an individual at risk for, suspected of having a sleep disorder, or being treated for a sleep disorder may be diagnosed by calculating the number of fold differences (i.e. 2-fold, 3-fold, etc.) between the measured biomarker value(s) in the individual and the reference value. A fold difference can additionally be a value in the range of 10% to 90% of the biomarker reference value. In other embodiments, a fold difference can be determined by measuring the absolute concentration of a biomarker and comparing that to the absolute value of a reference. Alternately, a fold difference can be measured as the relative difference between a biomarker reference value and a biomarker sample value, where neither value is a measure of absolute concentration, and/or where both values are measured simultaneously. In other embodiments, the fold difference between the measured biomarker value in the individual and the biomarker reference value may be compared to a minimum fold difference. In additional embodiments the measured levels of a biomarker(s) in a particular individual may be normalized against biomarker values from normal, healthy individuals. In further embodiments, the measured ranges and/or levels of a biomarker for a particular individual may be compared to reference ranges and/or levels for healthy individuals and reference ranges and/or levels of individuals previously diagnosed with sleep disorders.

For example, a baseline level of salivary 1-endorphin, chromogranin A, Annexin 1, BDNF, 3-methyl-2-oxobutyrate, creatine, tyrosine, arginine, dihomo-linolenate and linolenate is referred to the average level of salivary β-endorphin, chromogranin A, Annexin 1, BDNF, 3-methyl-2-oxobutyrate, creatine, tyrosine, arginine, dihomo-linolenate and linolenate in a particular population, measured when the population is well-rested, such that an elevated level of salivary β-endorphin, chromogranin A, Annexin 1, BDNF, 3-methyl-2-oxobutyrate, creatine, tyrosine, arginine, dihomo-linolenate and linolenate of an individual of the population, compared to the baseline level of these biomarkers for the population when well-rested, indicates sleepiness, sleeping disorder and/or identification of REM and NREM sleep loss/disturbances. Methods of measuring the subject in a well-rested individual are known in the art, and include the Stanford sleepiness scale.

In some embodiments, the biomarkers may be part of a biochip assay, a composition generally comprising a solid support or substrate to which a capture binding ligand is attached that can bind to proteins. Detection of a target species in some embodiments requires a label or detectable marker that can be incorporated as generally known to those of skill in the art. Such labels may be isotopic labels; magnetic, electrical or thermal labels; colored or luminescent dye; or enzymes, all of which enable detection of the biomarkers. In various embodiments, a secondary detectable label is used. A secondary label is one that is indirectly detected including, but not limited to, one of a binding partner pair; chemically modifiable moieties; nuclease inhibitors; enzymes such as horseradish peroxidase, alkaline phosphatases, luciferases etc. In sandwich formats of the invention, an enzyme serves as the secondary label, bound to the soluble capture ligand. In various embodiments, the system relies on detecting the precipitation of a reaction product or on a change on the properties of the label, for example the color for detection. A detection system for colorimetric methods includes any device that can be used to measure colorimetric properties. Generally, the device is a spectrophotometer, a colorimetcr, or any device that measures absorbance or transmission of light on one or more wavelengths.

Individuals diagnosed with sleepiness may be treated in myriad ways known to those of skill in the art including, but not limited to, a continuous positive airway pressure (CPAP) machine; exercise; diet changes; sleep hygiene; light phase shift therapy; cognitive behavioral therapy such as sleep restriction, stimulus control therapy, sleep hygiene, sleep environment improvement, relaxation training, paradoxical intention and biofeedback; pharmacological therapy such as, but not limited to, barbiturates, chloral hydrate, zolpidem, zaleplon, tasimelteon, suvorexant, benzodiazepines, temazepam, triazolam, estazolam, quazepam, flurazepam, escopiclone, melatonin receptor agonists, antihistamines, and antidepressants such as trazedone; and alternative substances such as valerian, skullcap, passionflower, chamomile and melatonin. Such treatment options may be used alone or together, by a coordinate method, administered simultaneously or sequentially or combinatorially formulated agents for the treatment of sleepiness.

Efficacy of the coordinate treatment methods and drug compositions of the invention will often be determined by measuremeits of biomarkers as well as use of conventional patient surveys or clinical scales to measure clinical indices of disorders in subjects. The methods and compositions of the invention will yield a reduction in one or more levels, scores or selected values generated from such measurements, surveys or scales completed by test subjects (indicating for example an incidence or severity of a selected sleep disorder or a secondary indication of a sleep disorder), by at least 10%, 20%, 30%, 50% or greater, up to 75-90%, or 95% compared to correlative scores or values observed for control subjects treated with placebo or other suitable control treatment. In at risk populations, the methods and compositions of the invention will yield a stable or minimally variable change in one or more levels, scores or selected values generated from such surveys or scales completed by test subjects. More detailed data regarding efficacy of the methods and compositions of the invention can be determined using alternative clinical trial designs.

The amount, timing and mode of delivery of compositions for the treatment of sleepiness comprising an effective amount of a therapeutic compound of one or more treatment options will be routinely adjusted on an individual basis, depending on such factors as weight, age, gender, and condition of the individual, the acuteness of the targeted anxiety disorder and/or related symptoms, whether the administration is prophylactic or therapeutic, and on the basis of other factors known to effect drug delivery, absorption, and pharmacokinetics, including half-life, and efficacy.

Effective unit dosage amounts of either or both of the pharmacological and alternative treatment options may be administered in a single dose, or in the form of multiple daily, weekly or monthly doses, for example in a dosing regimen comprising from 1 to 5, or 2-3, doses administered per day, per week, or per month. In exemplary embodiments, exemplary dosages of selected drugs as illustrated above are administered one, two, three, or four times per day. In more detailed embodiments, specific dosages within the specified exemplary ranges above are administered once, twice, or three times daily. In alternate embodiments, dosages are calculated based on body weight, and may be administered, for example, in amounts as exemplified above adjusted for body weight.

The present technology further provides a kit for diagnosing, monitoring and predicting sleepiness. The kit includes: (1) a composition or panel of any one or more of the above identified biomarkers; (2) a substrate for holding a biological sample isolated from a human subject suspected of having sleepiness, being at risk for sleepiness, or early detection, diagnosis and being under treatment for sleepiness; (3) an agent that binds to at least one of the biomarkers; (4) a detectable label such as one conjugated to the agent, or one conjugated to a substance that specifically binds at least one or more of the biomarkers and provides a proportional response based on the level of biomarker present and (5) printed instructions for reacting the agent with the biological sample or a portion of the biological sample to detect the presence or amount of at least one marker in the biological sample and determining if the marker is within a reference level of the biomarker. For example, a kit may comprise a saliva sample obtained from the patient; a plurality of test strips, each configured to produce a fluorescence level proportional to a level present on the test strip of one of a group of biomarkers; and a reading device configured to read the fluorescence levels on each of the test strips after the test strips are exposed to the saliva sample and wherein when the fluorescence levels indicate that two or more of the biomarkers are between a reference level the patient is determined to have sleepiness. In some embodiments, the kit may be used at the same time of day, in the same manner and/or with the same test used to determine the reference levels.

The terminology used herein is for the purpose of describing particular embodiments only and is not intended to be limiting of the invention. As used in this document, the singular forms “a,” “an,” and “the” include plural references unless the context clearly dictates otherwise. Unless defined otherwise, all technical and scientific terms used herein have the same meanings as commonly understood by one of ordinary skill in the art.

The present invention will now be described in greater detail with reference to the following examples without limiting the scope of the invention where tests were conducted on humans.

### EXAMPLES

### Example I

### Salivary Metabolome in Sleep

Thirty healthy male volunteers without any systemic diseases and with the demographic parameters shown in Table 1 were selected. The study conformed to the Declaration of Helsinki and had written informed consent.

TABLE 1
Demographics for Study

| N | 30 | |
|---|---|---|
| Age (years) | 54.3 | (1.2) |
| Body Mass Index | 25.4 | (1.3) |
| Pittsburgh Sleep Quality Index | 2.3 | (0.4) |
| Habitual Sleep Time (h) | 23.5 | (0.6) |
| Habitual Wake Time (h) | 7.3 | (0.5) |
| Munich Chronotype Questionnaire | 3.5 | (0.5) |
| Horne-Ostberg Questionnaire | 62.3 | (3.6) |

Before starting the study, each subject completed a general medical questionnaire, Quality of Life questionnaire, Pittsburgh sleep quality index, Epworth sleepiness scale, IQ questionnaire, Hamilton depression rating scale, atypical depression supplement and insomnia severity index questionnaire (Zavada A, Gordijn M C, Beersma D G, Daan S, Roenneberg T. Comparison of the Munich Chronotype Questionnaire with the Horne-Ostberg's Morningness-Eveningness Score. Chronobiol Int. 2005; 22(2):267-78; Buysse D J, Reynolds C F 3rd, Monk T H, Berman S R, Kupfer D J. The Pittsburgh Sleep Quality Index: a new instrument for psychiatric practice and research. Psychiatry Res. 1989 May; 28(2): 193-213; Johns M W. A new method for measuring daytime sleepiness: The Epworth Sleepiness Scale. Sleep. 1991; 14: 540-545; Schwab J J, Bialow M, Brown J M, Holzer C E. Diagnosing depression in medical inpatients. Ann Intern Med. 1967 October; 67(4):695-707; Bastien C H, Vallières A, Morin C M. Validation of the Insomnia Severity Index as an outcome measure for insomnia research. Sleep Med. 2001 July; 2(4):297-307; Viola A U, Tobaldini E, Chellappa S L, Casali K R, Porta A, Montano N. Short-term complexity of cardiac autonomic control during sleep: REM as a potential risk factor for cardiovascular system in aging. PLoS One. 2011 Apr. 22; 6(4):e19002).

Volunteers were excluded if they smoked, suffered from alcoholism or drug addiction, were on other medication, had performed shift work within the last 6 months, had a sleep efficiency <70%, >10 periodic leg movements per hour, or an apnea hypopnea index >10. Two weeks before the start of constant routine study, subjects recorded their sleep and wake time using actigraphy and sleep diaries. Habitual sleep times were estimated after two weeks and subjects were instructed to sleep according to habitual times during the week prior to the beginning of the study. Compliance was monitored by actigraphy and a sleep diary during the test week. A baseline night of sleep was taken prior to the start of the study followed by 40 h of prolonged wakefulness and then a recovery night as described in Czeisler C, Brown E, Ronda J, Kronauer R (1985) A clinical method to assess the endogenous circadian phase (ECP) of the deep circadian oscillator in man. J Sleep Res 14:295.

During the baseline tests, subjects were in a semi-reclining position in bed in a room with constant illumination level (<8 1×) and temperature (22° C.). Isocaloric meals and water were given once every hour during the awake period. A sociologist prevented the subjects from falling asleep during the 40 h of wakefulness. Hourly samples of saliva were taken during the period of extended wakefulness. Unstimulated saliva samples were collected at the start of the constant wakefulness period and every 4 hours by using the drooling method. The samples were centrifuged before freezing at −20° C. until analysis. Salivary β-endorphin, chromogranin-A, Annexin 1 and BDNF were measured using commercial kits. Small-Molecule Determination. Metabolites in saliva were analyzed by Metabolon, as described previously (Lawton K A, Berger A, Mitchell M, Milgram K E, Evans A M, Guo L, Hanson R W, Kalhan S C, Ryals J A, Milburn M V. Analysis of the adult human plasma metabolome. Pharmacogenomics. 2008 April; 9(4):383-97; Takeda I, Stretch C, Barnaby P, Bhatnager K, Rankin K, Fu H, Weljie A, Jha N, Slupsky C. Understanding the human salivary metabolome. NMR Biomed. 2009 July; 22(6):577-84).

The total process variability of metabolites was calculated as the relative standard variation of six runs of equal aliquots from each of the experimental samples. The variation among these six replicates was 9% in saliva. As a control, internal standards were injected into each of the samples and the variability was 6% in saliva. Raw peak values for all metabolites were normalized to have a mean median of I as the absolute level and thus the lower limit of detection was unknown. Metabolites were assessed using an algorithm (Hughes M E, Hogenesch J B, Kornacker K (2010) JTK CYCLE: An efficient nonparametric algorithm for detecting rhythmic components in genome-scale data sets. J Biol Rhythms 25:372-380). Pearson correlation coefficients and significance levels were computed using Prism 5 (GraphPad Software, La Jolla, Calif.).

The GC/LC-MS measurement procedure was independently validated with separate ELISA analyses to determine the subjects' β-endorphin, chromogranin A, Annexin I and BDNF levels. The pattern of β-endorphin, chromogranin A, Annexin 1 and BDNF levels was highly correlated between ELISA and GC/LC-MS (Pearson's r=0.74, 0.78, 0.78, 0.73, 0.74 P<0.0001 respectively), confirming the validity of the GCILC-MS quantification. 160 metabolites were detected in saliva. The largest groups of compounds in saliva were amino acids (3-methyl-2-oxobutyrate, creatine, tyrosine, and arginine), and fatty acids (dihomo-linolenate and linolenate). β-endorphin, chromogranin-A, Annexin 1 and BDNF levels were significantly correlated with sleep.

### Example II

### Salivary β-Endorphin (A), Chromogranin A (B), Annexin (C), BDNF (D), 3-Methyl-2-Oxobutyrate (E), Creatine (F), Tyrosine (G), Arginine (H), Dihomo-Linolenate (I) and Linolenate (J) as Biomarkers of Sleepiness in Patients

The subjects selected for this study were the same as those defined in Example 1. Unstimulated whole saliva specimens were collected and analyzed for salivary β-endorphin, chromogranin A, Annexin 1, BDNF, 3-methyl-2-oxobutyrate, creatine, tyrosine, arginine, dihomo-linolenate and linolenate biomarkers as for Example 1 above. Statistical comparison of 40 hour awake patients and health normal patients was performed using two-tailed t-test using GraphPad Prism for Windows, v 5.01 (GraphPad Software, La Jolla, Calif.). Receiver operating characteristic curves (ROC) were generated using R (R Foundation for Statistical Computing, Vienna, Austria).

TABLE 2
ROC Analysis and diagnostic performance of salivary β-endorphin (A), chromogranin-A
(B), Annexin (C), BDNF (D), 3-methyl-2-oxobutyrate (E), creatine (F), tyrosine (G), arginine
(H), dihomo-linolenate (I) and linolenate (J) biomarkers in sleepiness patients.

| Parameters | A | B | C | D | E | F | G | H | I | J |
|---|---|---|---|---|---|---|---|---|---|---|
| ROC AUC | 0.78 | 0.75 | 0.73 | 0.73 | 0.65 | 0.63 | 0.62 | 0.61 | 0.62 | 0.61 |
| Reference | 1491-2100 | 3.4-5.0 | 56-84 | 502-679 | 53-71.8 | 0.48-0.75 | 0.58-0.85 | 0.32-0.65 | 35-43 | 37-52 |
| | pg/ml | pmol/mg | ng/ml | pg/ml | μmol/ml | μmol/ml | μmol/ml | μmol/ml | μmol/ml | μmol/ml |
| | | protein | | | | | | | | |
| Sensitivity | 86 | 85 | 83 | 75 | 63 | 62 | 61 | 61 | 61 | 62 |

ROC analysis established good diagnostic sensitivity and specificity in sleepiness (Table 2) indicating that salivary β-endorphin, chromogranin A, Annexin 1, BDNF, 3-methyl-2-oxobutyrate, creatine, tyrosine, arginine, dihomo-linolenate and linolenate biomarkers have high values for the diagnosis of sleepiness.

### Example III

### Salivary Biomarkers of Sleepiness

Twenty-five age-matched healthy human adult volunteers (13 men and 12 women), were enrolled in the study after obtaining their consent. The study was approved by the Institutional Review Board. The demographics of the 25 adults were as shown in Table 3.

TABLE 3
Demographics for individuals in study

| N | 25 (13 men and 12 women) | |
|---|---|---|
| Age (years) | 56.5 | (3.4) |
| Body Mass Index | 24.6 | (1.6) |
| Pittsburgh Sleep Quality Index | 2.4 | (0.5) |
| Habitual Sleep Time (h) | 24.6 | (0.7) |
| Habitual Wake Time (h) | 6.6 | (0.4) |
| Munich Chronotype Questionnaire | 3.4 | (0.7) |
| Horne-Ostberg Questionnaire | 66.4 | (4.2) |

The subjects were randomly separated into two groups which were scheduled to alternate 2 weekends of either normal sleep or 28 hours of continuous waking. The sleep protocol was carried out in a standardized way (Effectiveness of portable monitoring devices for diagnosing obstructive sleep apnea: update of a systematic review. Prepared by RTI-UNC Evidence-based practice center. Research Triangle Park, North Carolina 27709 for Agency for Healthcare Research and Quality, U.S. Department of Health and Human Services, 2004. Standards for Accreditation of Sleep Disorders Centers. American Academy of Sleep Medicine, Westchester, Ill. 60154 2007). On the normal sleep weekend, the volunteers were allowed to fall asleep at 9:00 pm. Normal sleep architecture and absence of significant respiratory abnormalities during sleep, periodic limb movement disorder parasomnias, and nocturnal seizures were confirmed by standard polysomnography. The polysomnograms were evaluated and scored following standard criteria (Rechtschaffen, A. and Kales, A., editors (1968)). A manual of standardized terminology, techniques and scoring system for sleep stages in human subjects were carried out. The sleep deprivation group remained awake and was allowed free access to water during the night. However, meal times were restricted to 7 am, 12:30 pm, and 7 pm. No efforts were taken to limit caffeine consumption during the day and logs were kept for each participant. The participants were constantly monitored by three experienced experts. Unstimulated saliva was collected three times per day. (early morning, afternoon and night) (Rai B, Kaur J, Anand S C, Jacobs R. Salivary stress markers, stress, and periodontitis: a pilot study. J Periodontol. 2011 February; 82(2):287-92).

Care was taken to ensure that all samples were taken at the same time of day and in the same manner. Individuals were asked to abstain from eating and drinking for at least two hours prior to sample collection. Both stimulated and unstimulated samples were collected. Ten minutes prior to collection of unstimulated saliva samples, individuals were asked to rinse orally with water. At the time of sample collection, study members were asked to relax for 5-15 minutes. They were then seated in a bent forward position in an ordinary chair and asked to put their tongues on the lingual surfaces of the upper incisors and allow the saliva to drip into sterile plastic (glass) tubes treated with 50 g of 2% sodium azide solution to prevent microbial decomposition of saliva. The tubes were held to the lower lip for 10 minutes resulting in a collection of 1-5 ml of saliva per individual. Stimulated samples were collected ten minutes after the unstimulated samples using the Salivette® polyester roll device.

The samples were rapidly frozen over dry ice and kept at −80° C. until analyzed. Salivary β-endorphin, chromogranin A, Annexin 1, BDNF, 3-methyl-2-oxobutyrate, creatine, tyrosine, arginine, dihomo-linolenate and linolenate were measured by ELISA using commercial kits (β-endorphins-1134 kit, peninsula Laboratories Inc., Belmont, Calif., USA; YKO70 Human CgA EIA kit, Quantikine Human BDNF immunoassay (R&D; System, Minneapolis, Minn., USA), EIA (Salimetrics, UK), respectively.

While salivary β-endorphin, chromogranin A, Annexin 1, BDNF, 3-methyl-2-oxobutyrate, creatine, tyrosine, arginine, dihomo-linolenate and linolenate levels were non-significantly lower in stimulated saliva as compared to unstimulated saliva in both groups, as shown in Table 3, salivary β-endorphin, chromogranin A, Annexin 1, BDNF, 3-methyl-2-oxobutyrate, creatine, tyrosine, arginine, dihomo-linolenate and linolenate were significantly changed in day time and night time in the continuous waking group in comparison to the normal sleep group (Table 4).

TABLE 4
Salivary β-endorphin, chromogranin-A, Annexin 1 and BDNF levels in normal sleep subjects and continuously awake subjects.

| | Normal sleep | | | | | | Continuously Awake | | | | | |
|---|---|---|---|---|---|---|---|---|---|---|---|---|
| | Mean (standard deviation) | | | | | | Mean (standard deviation) | | | | | |
| | | | | | Evening | | | | | | Evening | |
| Parameters | Morning | | Afternoon | | (night) | | Morning | | Afternoon | | (night) | |
| CgA (pmol/mg protein) | 2.98 | (0.62) | 3.02 | (0.45) | 3.12 | (0.65) | 3.89 | (0.56) | 4.35 | (0.34) | 4.98 | (0.24) |
| B-endorphin (pg/mL) | 1285.8 | (62.3) | 1362.3 | (75.4) | 1412.6 | (67.3) | 1543.7 | (78.9) | 1678.9 | (72.4) | 2002.3 | (97.2) |
| BDNF (pg/ml) | 432.2 | (45.2) | 345.4 | (40.5) | 234.3 | (34.1) | 634.5 | (45.2) | 478.5 | (42.5) | 367.6 | (43.4) |
| Annexin 1 (ng/ml) | 48.6 | (6.2) | 34.6 | (5.3) | 12.5 | (3.6) | 78.8 | (5.3) | 56.7 | (5.3) | 54.8 | (4.3) |
| Arginine (μmol/ml) | 0.22 | (0.02) | 0.31 | (0.03) | 0.24 | (0.02) | 0.45 | (0.04) | 0.56 | (0.03) | 0.64 | (0.05) |
| Tyrosine (μmol/ml) | 0.34 | (0.05) | 0.56 | (0.05) | 0.43 | (0.04) | 0.67 | (0.03) | 0.78 | (0.04) | 0.84 | (0.03) |
| Creatine (μmol/ml) | 0.43 | (0.04) | 0.45 | (0.03) | 0.46 | (0.05) | 0.63 | (0.03) | 0.65 | (0.02) | 0.74 | (0.06) |
| 3-methyl-2- | 34.4 | (9.5) | 37.5 | (8.4) | 43.4 | (7.4) | 48.4 | (6.8) | 62.4 | (6.9) | 65.4 | (6.4) |
| oxobutyrate (μmol/ml) | | | | | | | | | | | | |
| Dihomo-linolenate (μmol/ml) | 23.1 | (3.3) | 24.2 | (4.5) | 26.4 | (6.6) | 34.2 | (3.7) | 36.4 | (4.5) | 38.3 | (4.8) |
| Linolenate (μmol/ml) | 23.3 | (4.5) | 24.3 | (6.3) | 27.5 | (6.8) | 39.4 | (6.6) | 43.6 | (4.2) | 46.7 | (5.6) |

### Example IV

### Salivary β-Endorphin, Chromogranin A, Annexin 1, BDNF, 3-Methyl-2-Oxobutyrate, Creatine, Tyrosine, Arginine, Dihomo-Linolenate and Linolenate in the Determination of Sleep Quality in Depressed Patients Compared to Normal Healthy Subjects

This study comprised 65 patients with minor depression complaining of insomnia. Patients with psychotic disorder, bipolar disorder, chronic alcoholism, dementia or organic brain disorders were not included in the study. The diagnosis of depression was based on Structured Clinical Interview according to DSM III. Special attention was paid to the occurrence and intensity of anxiety symptoms. Stable dosages of anxiety medication (two weeks or more) were continued. Electroencephalogram (EEG), electrooculogram (EOG), electromyogram (EMG) and electrocardiogram (ECG) readings taken during sleep and after waking were used to diagnose insomnia according to DSM III criteria.

Individuals were asked to abstain from eating or drinking for at least two hours prior to sample collection. Ten minutes prior to collection of unstimulated saliva samples, individuals were asked to rinse orally with water. Samples were collected in the morning, the afternoon and evening. At the time of sample collection, study members were asked to relax for 5-15 minutes. They were then seated in a bent forward position in an ordinary chair and asked to put their tongues on the lingual surfaces of the upper incisors and allow the saliva to drip into sterile plastic (glass) tubes treated with 50 g of 2% sodium azide solution to prevent microbial decomposition of saliva. The tubes were held to the lower lip for 10 minutes resulting in a collection of 1-5 ml of saliva per individual. Salivary 1-endorphin, chromogranin-A, BDNF, Annexin 1 were measured using commercial kits (β-endorphin s-1134 kit, peninsula Laboratories Inc., Belmont, Calif., USA; YKO70 Human CgA EIA kit, Quantikine Human BDNF immunoassay (R&D; System, Minneapolis, Minn., USA), EIA (Salimetrics, UK). 3-methyl-2-oxobutyrate, creatine, tyrosine, arginine, dihomo-linolenate and linolenate were measured using NMR and compared to normal sleep subjects.

As shown in Table 5, salivary β-endorphin, chromogranin A, Annexin 1, BDNF, 3-methyl-2-oxobutyrate, creatine, tyrosine, arginine, dihomo-linolenate and linolenate levels were significantly different than those of normal healthy adults (p=0.0001).

TABLE 5
Comparison of Biomarker Levels in Depressed vs. Normal Individuals

| | | | | | | | Depressed Patients | |
|---|---|---|---|---|---|---|---|---|
| | Normal sleep | | | | | | Average of | |
| | | | | | Evening | | Morning/afternoon/ | |
| Parameters | Morning | | Afternoon | | (night) | | evening measurements | |
| CgA (pmol/mg protein) | 2.98 | (0.62) | 3.02 | (0.45) | 3.12 | (0.65) | 4.1 | (0.6) |
| B-endorphin (pg/mL) | 1285.8 | (62.3) | 1362.3 | (75.4) | 1412.6 | (67.3) | 1672 | (12.1) |
| BDNF (pg/ml) | 432.2 | (45.2) | 345.4 | (40.5) | 234.3 | (34.1) | 612 | (11.3) |
| Annexin 1 (ng/ml) | 48.6 | (6.2) | 34.6 | (5.3) | 12.5 | (3.6) | 72 | (9) |
| Arginine (μmol/ml) | 0.22 | (0.02) | 0.31 | (0.03) | 0.24 | (0.02) | 0.54 | (0.1) |
| Tyrosine (μmol/ml) | 0.34 | (0.05) | 0.56 | (0.05) | 0.43 | (0.04) | 0.72 | (0.05) |
| Creatine (μmol/ml) | 0.43 | (0.04) | 0.45 | (0.03) | 0.46 | (0.05) | 0.63 | (0.06) |
| 3-methyl-2-oxobutyrate (μmol/ml) | 34.4 | (9.5) | 37.5 | (8.4) | 43.4 | (7.4) | 68.2 | (11.3) |
| Dihomo-linolenate (μmol/ml) | 23.1 | (3.3) | 24.2 | (4.5) | 26.4 | (6.6) | 34.2 | (3.4) |
| Linolenate (μmol/ml) | 23.3 | (4.5) | 24.3 | (6.3) | 27.5 | (6.8) | 48 | (2.3) |

### Example V

### Salivary β-Endorphin, Chromogranin A, Annexin 1, BDNF, 3-Methyl-2-Oxobutyrate, Creatine, Tyrosine, Arginine, Dihomo-Linolenate and Linolenate as Diagnostic, Prognostic and Monitoring Biomarkers for Obstructive Sleep Apnea Patients

Subjects were 22 volunteer patients (10 males, 12 females, aged 37-74 years) referred for polysomnography (PSG) testing. Subjects were excluded if they had cardiovascular complications, orthopedic disabilities, or recent participation in moderately vigorous physical activity (Strenuous enough to burn off three to six times as much energy per minute as sitting quietly). Each subject underwent PSG testing to confirm the presence of Obstructive Sleep Apnea. Before treatment, all subjects performed a cycle ergometer test, using a ramp protocol (graded exercise test in which the treadmill speed is kept constant, but the grade increases each minute between 1% and 4% until volitional exhaustion or other test termination criteria are achieved) designed to achieve 75% of VO₂max in 17+2 minutes. The subjects performed the same test 7 days later, and again after 4 weeks of CPAP therapy. Respiratory gas exchange, electrocardiograph, blood pressure, rating of perceived exertion (RPE), and heart rate were monitored. Ten minutes prior to unstimulated saliva sample collection, subjects were asked to rinse orally with water. At the time of sample collection, study members were asked to relax for 5-15 minutes. They were then seated in a bent forward position in an ordinary chair and asked to put their tongues on the lingual surfaces of the upper incisors and allow the saliva to drip into sterile plastic (glass) tubes treated with 50 g of 2% sodium azide solution to prevent microbial decomposition of saliva. The tubes were held to the lower lip for 10 minutes resulting in a collection of 1-5 ml of saliva per individual. The presence of salivary β-endorphin, chromogranin-A, BDNF), Annexin 1 were measured using commercial kits (β-endorphin s-1134 kit, peninsula Laboratories Inc., Belmont, Calif., USA; YKO70 Human CgA EIA kit, Quantikine Human BDNF immunoassay (R&D; System, Minneapolis, Minn., USA), EIA (Salimetrics, UK) respectively. 3-methyl-2-oxobutyrate, creatine, tyrosine, arginine, dihomo-linolenate and linolenate were measured using NMR and the results were compared to the normal sleep subjects of Example III.

Results indicated that after treatment with CPAP, heart rates at 60% of the subject's age adjusted maximum were significantly lower (−10.2 beats/min, p=0.043). Heart rate and systolic blood pressure at rest were not different after training. RPE was significantly lower after training for similar workloads (p=0.04). In addition, individuals with the worst scores on the PSG tests (and therefore the most severe disease) showed the greatest reduction in RPE at similar workloads after training. Four weeks of CPAP therapy significantly increases aerobic fitness, as well as improved patient perception of sleep quality and physical vitality. A comparison of salivary β-endorphin, chromogranin A, Annexin 1, BDNF, 3-methyl-2-oxobutyrate, creatine, tyrosine, arginine, dihomo-linolenate and linolenate levels taken before and after treatment indicated significant changes as compared to normal (P=0001).

TABLE 6
Comparison of Biomarker Levels in Normal Health Individuals to Individuals
diagnosed with insomnia before and after treatment with a CPAP machine.

| | Normal sleep | | | | | | | | | |
|---|---|---|---|---|---|---|---|---|---|---|
| | Mean (standard deviation) | | | | | | Treatment with CPAP | | | |
| | | | | | Evening | | Before | | After | |
| Biomarker | Morning | | Afternoon | | (night) | | Treatment | | Treatment | |
| CgA (pmol/mg protein) | 2.98 | (0.62) | 3.02 | (0.45) | 3.12 | (0.65) | 13.56 | (2.3) | 6.13 | (2.13) |
| B-endorphin (pg/mL) | 1285.8 | (62.3) | 1362.3 | (75.4) | 1412.6 | (67.3) | 2105.9 | (567.4) | 1789.2 | (412.3) |
| BDNF (pg/ml) | 432.2 | (45.2) | 345.4 | (40.5) | 234.3 | (34.1) | 367.4 | (58.4) | 265.3 | (45.2) |
| Annexin 1 (ng/ml) | 48.6 | (6.2) | 34.6 | (5.3) | 12.5 | (3.6) | 71.3 | (13.1) | 51.2 | (15.2) |
| Arginine (μmol/ml) | 0.22 | (0.02) | 0.31 | (0.03) | 0.24 | (0.02) | 0.58 | (0.03) | 0.43 | (0.04) |
| Tyrosine (μmol/ml) | 0.34 | (0.05) | 0.56 | (0.05) | 0.43 | (0.04) | 0.62 | (0.01) | 0.45 | (0.03) |
| Creatine (μmol/ml) | 0.43 | (0.04) | 0.45 | (0.03) | 0.46 | (0.05) | 0.59 | (0.05) | 0.43 | (0.19) |
| 3-methyl-2-oxobutyrate (μmol/ml) | 34.4 | (9.5) | 37.5 | (8.4) | 43.4 | (7.4) | 62.1 | (4.3) | 53.2 | (3.1) |
| Dihomo-linolenate (μmol/ml) | 23.1 | (3.3) | 24.2 | (4.5) | 26.4 | (6.6) | 38.5 | (3.4) | 26.2 | (1.9) |
| Linolenate (μmol/ml) | 23.3 | (4.5) | 24.3 | (6.3) | 27.5 | (6.8) | 45.3 | (4.7) | 34.3 | (4.5) |

### Example VI

### Salivary β-Endorphin, Chromogranin A, Annexin 1, BDNF, 3-Methyl-2-Oxobutyrate, Creatine, Tyrosine, Arginine, Dihomo-Linolenate and Linolenate Biomarkers for Sleep Disturbances in Extreme Conditions and in Mars Analog Environments

This study was based on six participants in the Mars desert research station, USA (extreme conditions, isolated area, lots of noise, heavy workload) with complaints of insomnia. Insomnia was diagnosed using electroencephalogram (EEG), electrooculogram (EOG), electromyogram (EMG) and electrocardiogram (ECG). Unstimulated saliva samples were collected and biomarker levels measured. Ten minutes prior to collection of unstimulated saliva samples, individuals were asked to rinse orally with water. At the time of sample collection, study members were asked to relax for 5-15 minutes. They were then seated in a bent forward position in an ordinary chair and asked to put their tongues on the lingual surfaces of the upper incisors and allow the saliva to drip into sterile plastic (glass) tubes treated with 50 g of 2% sodium azide solution to prevent microbial decomposition of saliva. The tubes were held to the lower lip for 10 minutes resulting in a collection of 1-5 ml of saliva per individual. Salivary β-endorphin, chromogranin-A, BDNF, Annexin 1 were measured using commercial kits (β-endorphin s-1134 kit, peninsula Laboratories Inc., Belmont, Calif., USA; YKO70 Human CgA EIA kit, Quantikine Human BDNF immunoassay (R&D; System, Minneapolis, Minn., USA), EIA (Salimetrics, UK). 3-methyl-2-oxobutyrate, creatine, tyrosine, arginine, dihomo-linolenate and linolenate were measured using NMR and compared to normal sleep subjects of Example II.

Results: As shown in Table 7, salivary β-endorphin, chromogranin A, Annexin 1, BDNF, 3-methyl-2-oxobutyrate, creatine, tyrosine, argininc, dihomo-linolenate and linolenate levels were significantly changed as compared to normal healthy individuals of Table 4 (p=0.0001).

TABLE 7
Comparison of Normal Healthy individuals to individuals complaining of insomnia

| | Normal sleep | | | | | | | | | |
|---|---|---|---|---|---|---|---|---|---|---|
| | Mean (standard deviation) | | | | | | Individuals complaining of insomnia | | | |
| | | | | | Evening | | Prior to starting | | After finishing | |
| Parameters | Morning | | Afternoon | | (night) | | mission | | mission | |
| CgA (pmol/mg protein) | 2.98 | (0.62) | 3.02 | (0.45) | 3.12 | (0.65) | 3.21 | (0.34) | 3.9 | (0.5) |
| B-endorphin (pg/mL) | 1285.8 | (62.3) | 1362.3 | (75.4) | 1412.6 | (67.3) | 1561.3 | (78.4) | 1781.2 | (13.5) |
| BDNF (pg/ml) | 432.2 | (45.2) | 345.4 | (40.5) | 234.3 | (34.1) | 278.4 | (24.4) | 568.3 | (23.4) |
| Annexin 1 (ng/ml) | 48.6 | (6.2) | 34.6 | (5.3) | 12.5 | (3.6) | 13.1 | (7.9) | 56.3 | (11.2) |
| Arginine (μmol/ml) | 0.22 | (0.02) | 0.31 | (0.03) | 0.24 | (0.02) | 0.34 | (0.09) | 0.65 | (0.2) |
| Tyrosine (μmol/ml) | 0.34 | (0.05) | 0.56 | (0.05) | 0.43 | (0.04) | 0.41 | (0.05) | 0.73 | (0.04) |
| Creatine (μmol/ml) | 0.43 | (0.04) | 0.45 | (0.03) | 0.46 | (0.05) | 0.42 | (0.02) | 0.61 | (0.12) |
| 3-methyl-2-oxobutyrate (μmol/ml) | 34.4 | (9.5) | 37.5 | (8.4) | 43.4 | (7.4) | 41.2 | (6.3) | 72.3 | (10.6) |
| Dihomo-linolenate (μmol/ml) | 23.1 | (3.3) | 24.2 | (4.5) | 26.4 | (6.6) | 27.2 | (6.9) | 38.3 | (4.2) |
| Linolenate (μmol/ml) | 23.3 | (4.5) | 24.3 | (6.3) | 27.5 | (6.8) | 23.2 | (5.6) | 56.4 | (5.6) |

### Example VII

### Diagnostic Value of Salivary Biomarkers in Patterns and the Consequences of Inadequate Sleep in Medical Students

Postgraduates (20: 27 (male: female) aged 26.42 (2.6) years, completed Epworth Sleepiness Scale questionnaires and 1 week of daily sleep diaries. Students in medical psychiatry courses earned extra credit for completing the questionnaire and sleep diary. During final semesters, data collection ended prior to finals week so data were not influenced by final exams.

Subjects were asked, each morning upon awakening, to complete daily sleep diaries as shown in Table 8. Subjects also reported medication and alcohol used as sleep aids, and herbal stimulants used to increase alertness. Subjects reported ideal amount of sleep and minimum amount of sleep to function during the day.

TABLE 8
Sleep Diary

| | Parameters mean | |
|---|---|---|
| Sleep variables and salivary biomarkers | (standard deviation) | |
| Time in bed | 8 H 40 min | (83 min) |
| Total sleep time | 7 H 46 min | (56 min) |
| Sleep onset latency | 23.23 | (20) min |
| Awakenings | 0.89 | (0.70) |
| Wake time after sleep onset | 12 | (12) min |
| Time awake prior to rising | 17 | (14) min |
| Sleep efficiency (Epworth Sleepiness Scale) | 82.34 | (8.34)% |
| Nap time | 34 | (31) min |
| How did you feel during the day? (0-10) | 5.45 | (1.23) |
| How did you feel when you woke up this | 5.23 | (2.12) |
| morning? (0-10) | | |
| What was the quality of your sleep last | 6.34 | (1.23) |
| night?(0-10) | | |
| Weekday bedtime | 11:30 P.M. | (134) min |
| Weekday wake time | 8:00 A.M. | (89) min |
| Weekend bedtime | 1:30 A.M. | (130) min |
| Weekend wake time | 11:20 A.M. | (120) min |
| Ideal amount of sleep | 8 h 45 min | (45) min |
| Minimum amount of sleep needed to | 4 h 30 min | (78) min |
| function during the day. | | |

Subjects were additionally asked “have you ever fallen asleep while driving?” and “have you ever gotten in a motor vehicle accident due to sleepiness?” 80% of subjects indicated that they had experienced sleepiness.

Unstimulated saliva samples were collected and biomarker levels measured. Ten minutes prior to collection of unstimulated saliva samples, individuals were asked to rinse orally with water. At the time of sample collection (in the morning after study time), study members were asked to relax for 5-15 minutes. They were then seated in a bent forward position in an ordinary chair and asked to put their tongues on the lingual surfaces of the upper incisors and allow the saliva to drip into sterile plastic (glass) tubes treated with 50 g of 2% sodium azide solution to prevent microbial decomposition of saliva. The tubes were held to the lower lip for 10 minutes resulting in a collection of 1-5 ml of saliva per individual. Salivary β-endorphin, chromogranin-A, BDNF, Annexin I were measured using commercial kits (β-endorphin s-1134 kit, peninsula Laboratories Inc., Belmont, Calif., USA; YKO70 Human CgA EIA kit, Quantikine Human BDNF immunoassay (R&D; System, Minneapolis, Minn., USA), EIA (Salimetrics, UK). 3-methyl-2-oxobutyrate, creatine, tyrosine, arginine, dihomo-linolenate and linolenate were measured using NMR and compared to normal sleep subjects of Example II.

As shown in Table 9, salivary β-endorphin, chromogranin A, Annexin 1, BDNF, 3-methyl-2-oxobutyrate, creatine, tyrosine, arginine, dihomo-linolenate and linolenate levels were significantly higher as compared to normal healthy individuals. (p=0.0001)

TABLE 9
Comparison of Normal Individuals with Post Graduate Students.

| | | | Post Graduate Students | |
|---|---|---|---|---|
| | Normal sleep | | complaining of sleepiness | |
| Parameters | Morning | | Morning | |
| CgA (pmol/mg protein) | 2.98 | (0.62) | 7.98 | (0.96) |
| B-endorphin (pg/mL) | 1285.8 | (62.3) | 2432.4 | (245.8) |
| BDNF (pg/ml) | 432.2 | (45.2) | 734.8 | (98.4) |
| Annexin 1 (ng/ml) | 48.6 | (6.2) | 76.8 | (12.3) |
| Arginine (μmol/ml) | 0.22 | (0.02) | 0.52 | (0.01) |
| Tyrosine (μmol/ml) | 0.34 | (0.05) | 0.71 | (0.07) |
| Creatine (μmol/ml) | 0.43 | (0.04) | 0.61 | (0.04) |
| 3-methyl-2-oxobutyrate | 34.4 | (9.5) | 66.2 | (8.4) |
| (μmol/ml) | | | | |
| Dihomo-linolenate | 23.1 | (3.3) | 34.4 | (3.6) |
| (μmol/ml) | | | | |
| Linolenate (μmol/ml) | 23.3 | (4.5) | 43 | (3.4) |

### Example VIII

### Role of Salivary Biomarkers in Monitoring of Sleepiness in Patients on a Sleep Disorder Treatment Regimen

In a hospital based clinical trial of zolpidem, zolpidem was administered to 10 patients diagnosed with idiopathic central sleep apnea. Idiopathic central sleep apnea was defined by central apnea and hypopnea index (CAHI)≧10 events per hour of sleep, and obstructive apnea/hypopnea index ≦5 events per hour of sleep. An apnea was defined as a cessation or reduction in peak oronasal airflow≧70% of baseline, lasting ≧15 s. Hypopnea was defined as a reduction in peak oronasal airflow ≧50%, ≧70% of baseline for ≧15 s, along with a ≧4% drop in SpO₂ relative to pre-event baseline value. The apnea/hypopnea index was calculated for all events relative to total sleep time and documented separately for central and obstructive events. Central apneas and hypopneas comprised the central and obstructive apneas and hypopneas plus mixed apneas and hypopneas comprised the obstructive apnea-hypopnea index (OAHI). Exclusion criteria comprise such as clinical or echocardiographic diagnosis of systolic heart failure, diastolic cardiac dysfunction, or a history of transient ischemic attack or stroke. Those with a typical pattern of Cheyne Stokes respiration during sleep were also excluded. Patients with obstructive sleep apnea with an Apnea-Hypopnea Index of (AHI) ≧5 events per hour prior to continuous positive airway pressure device (CPAP) use, or a history of restless legs syndrome/periodic leg movements were also excluded. Patients with prior use of sedative agents such as zolpidem, acetazolamide, theophylline, medroxyprogesterone, or any opiate, were also excluded from the study to exclude comparative bias when evaluating subjective response to zolpidem. Patients with hypercapnic lung disease or those with obesity hypoventilation syndrome were also excluded. Subjects were given the Epworth Sleepiness Scale (ESS), and a physical exam followed by an 8-h diagnostic polysomnogram (PSG). Patients were diagnosed with sleep apnea if they had excessive daytime sleepiness (ESS score ≧10 and had a CAHI ≧10 events/hour and an obstructive apnea index <5 events/hour. Within one week of the polysomnogram, and without any intervening treatment, patients were offered zolpidem treatment after reviewing the potential benefits, risks, and known outcomes of this therapy and of alternative therapies: oxygen, CPAP, bilevel pressure ventilation, and acetazolamide. Patients who consented to take zolpidem were instructed to take 10 mg orally, approximately 45 minutes before bedtime each night, including the night of the follow-up polysomnogram. Outpatient monitoring was conducted every four weeks to assess patients for side effects and for evaluation of daytime sleepiness, using the ESS. Six to ten weeks following zolpidem initiation, a repeat nighttime 8 hour PSO was scheduled to assess the effects of treatment. Nocturnal polysomnographic assessment using the Grass Aurora system was performed in a standard way consisting of an electroencephalogram (EEG), an electrooculogram (EOG), a submental electromyogram (EMG), an anterior tibialis EMG, and an electrocardiogram (ECG) (Rechtschaffen A, Kales A. A manual of standardized terminology, techniques and scoring system for sleep stages in human subjects. Washington D.C.: US Government Printing Office. National Institute of Health Publication; 1968). Nasal-oral airflow was measured using thermocouples. Abdominal and thoracic respiratory movements were recorded with respiratory effort belts using piezoelectric crystal sensors. Transcutaneous oxyhemoglobin saturation (SpO₂) was monitored with finger pulse oximetry. A recording of sleep position was performed using video monitoring and snoring was recorded using a snore sensor. An experienced sleep physician, blinded to the study protocol, scored sleep recordings. Sleep architecture was analyzed according to standardized way (Rechtschaffen A, Kales A. A manual of standardized terminology, techniques and scoring system for sleep stages in human subjects. Washington D.C.: US Government Printing Office. National Institute of Health Publication; 1968). Unstimulated saliva samples were collected and biomarker levels measured in the morning prior to beginning and after 150 days of study. Ten minutes prior to collection of unstimulated saliva samples, individuals were asked to rinse orally with water. At the time of sample collection, study members were asked to relax for 5-15 minutes. They were then seated in a bent forward position in an ordinary chair and asked to put their tongues on the lingual surfaces of the upper incisors and allow the saliva to drip into sterile plastic (glass) tubes treated with 50 g of 2% sodium azide solution to prevent microbial decomposition of saliva. The tubes were held to the lower lip for 10 minutes resulting in a collection of 1-5 ml of saliva per individual. Salivary β-endorphin, chromogranin-A, BDNF, Annexin 1 were measured using commercial kits (β-endorphins-1134 kit, Peninsula Laboratories Inc., Belmont, Calif., USA; YKO70 Human CgA EIA kit, Quantikine Human BDNF immunoassay (R&D; System, Minneapolis, Minn., USA), EIA (Salimetrics, UK) respectively. 3-methyl-2-oxobutyrate, creatine, tyrosine, arginine, dihomo-linolenate and linolenate were measured using NMR and compared to the normal sleep subjects of Example III.

A follow-up polysomnogram showed that the overall apnea/hypopnea index and central AHI (CAHI) significantly decreased. Also, the total number of arousals per hour decreased with treatment use, leading to a significant improvement in sleep efficiency. There was a positive correlation between the decrease in CAHI and the arousal index. Consistent with the hypnotic effect of zolpidem, sleep latency decreased, stage 1, 3, and 4 sleep percentages decreased, and stage 2 percentages increased. Excessive daytime sleepiness, measured by the Epworth Sleepiness Scale (ESS) decreased as shown in Table 10.

TABLE 10
Excessive daytime sleepiness as measured
by the Epworth Sleepiness Scale

| | | Range | | P value |
|---|---|---|---|---|
| Apnea hypopnea | Before | 32.6 | (13.5) | 0.0001 |
| | After | 12.6 | (13.1) | |
| Central Apnea | Before | 27.8 | (13.5) | 0.0001 |
| hypopnea Index | After | 6.8 | (5.8) | |
| Obstructive Apnea | Before | 4.2 | (2.8) | 0.05 |
| Hypopnea index | After | 6.8 | (1.6) | |
| Arousal index | Before | 23.7 | (10.8) | 0.0001 |
| | After | 14.3 | (12.2) | |
| Epworth sleepiness | Before | 13.7 | (3.7) | 0.0001 |
| score | After | 6.5 | (3.7) | |
| Obstructive Apnea | Before | 0.8 | (1.3) | 0.05 |
| index | After | 1.8 | (2.3) | |
| Obstructive hypopnea | Before | 2.1 | (0.9) | 0.001 |
| index | After | 4.5 | (1.3) | |
| Central Apnea index | Before | 13.5 | (12.7) | 0.05 |
| | After | 2.3 | (2.1) | |
| Central hypopnea | Before | 13.6 | (4.7) | 0.002 |
| index | After | 4.2 | (4.6) | |
| Total sleep time | Before | 4.6 | (1.4) | 0.005 |
| (hours) | After | 6.3 | (1.8) | |
| Sleep efficiency (%) | Before | 70.2 | (17.6) | 0.0001 |
| | After | 85.3 | (12.3) | |
| % TST stage 1 | Before | 34.6 | (13.5) | 0.05 |
| | After | 21.3 | (12.5) | |
| % TST stage 2 | Before | 42.2 | (13.6) | 0.001 |
| | After | 67.8 | (17.8) | |
| % TST stage 3 and 4 | Before | 1.3 | (2.3) | 0.05 |
| | After | 0.8 | (1.6) | |
| % TST REM | Before | 10.4 | (8.2) | 0.05 |
| | After | 14.5 | (6.9) | |

Also, salivary β-endorphin, chromogranin A, Annexin 1, I3DNF, 3-methyl-2-oxobutyrate, creatine, tyrosine, arginine, dihomo-linolenate and linolenate were significantly changed after treatment as shown in Table 11.

TABLE 11
Comparison of Biomarkers in Patients with Idiopathic Central Sleep Apnea on Zoldipam

| | | | | | | | Patients with idiopathic central sleep | | | | |
|---|---|---|---|---|---|---|---|---|---|---|---|
| | Normal sleep | | | | | | apnea given Zoldipam. | | | | |
| | | | | | Evening | | Before | | After | | |
| Biomarker | Morning | | Afternoon | | (night) | | Treatment | | Treatment | | |
| CgA (pmol/mg protein) | 2.98 | (0.62) | 3.02 | (0.45) | 3.12 | (0.65) | 13.68 | (3.23) | 4.23 | (2.31) | 0.0001 |
| B-endorphin (pg/mL) | 1285.8 | (62.3) | 1362.3 | (75.4) | 1412.6 | (67.3) | 2134.9 | (567.4) | 1235.3 | (452.3) | 0.0001 |
| BDNF (pg/ml) | 432.2 | (45.2) | 345.4 | (40.5) | 234.3 | (34.1) | 345.8 | (58.4) | 234.5 | (56.3) | 0.0001 |
| Annexin 1 (ng/ml) | 48.6 | (6.2) | 34.6 | (5.3) | 12.5 | (3.6) | 72.1 | (15.2) | 56.3 | (13.4) | 0.0001 |
| Arginine (μmol/ml) | 0.22 | (0.02) | 0.31 | (0.03) | 0.24 | (0.02) | 0.56 | (0.04) | 0.45 | (0.03) | 0.001 |
| Tyrosine (μmol/ml) | 0.34 | (0.05) | 0.56 | (0.05) | 0.43 | (0.04) | 0.67 | (0.05) | 0.54 | (0.04) | 0.005 |
| Creatine (μmol/ml) | 0.43 | (0.04) | 0.45 | (0.03) | 0.46 | (0.05) | 0.64 | (0.12) | 0.47 | (0.14) | 0.001 |
| 3-methyl-2- | 34.4 | (9.5) | 37.5 | (8.4) | 43.4 | (7.4) | 61.2 | (5.2) | 54.1 | (4.7) | 0.005 |
| oxobutyrate (μmol/ml) | | | | | | | | | | | |
| Dihomo-linolenate (μmol/ml) | 23.1 | (3.3) | 24.2 | (4.5) | 26.4 | (6.6) | 34.4 | (3.6) | 27.1 | (2.6) | 0.0001 |
| Linolenate (μmol/ml) | 23.3 | (4.5) | 24.3 | (6.3) | 27.5 | (6.8) | 36.5 | (4.1) | 21.2 | (1.8) | 0.0001 |

### Example IX

### Combination Biomarker Panel of Salivary β-Endorphin (A), Chromogranin A (B), Annexin (C), BDNF (D), 3-Methyl-2-Oxobutyrate (E), Creatine (F), Tyrosine (G), Arginine (H), Dihomo-Linolenate (I) and Linolenate (J) in Sleepiness Patients

The subjects were the same as those in Example IV. Unstimulated whole saliva specimens were collected and analyzed for salivary β-endorphin, chromogranin A, Annexin, BDNF, 3-methyl-2-oxobutyrate, creatine, tyrosine, arginine, dihomo-linolenate and linolenate as in Example I above. Statistical comparison of sleepiness by combination of salivary biomarkers was performed using the two-tailed t-test using GraphPad Prism for Windows, v 5.01 (GraphPad Software, San Diego, Calif.). Receiver operating characteristic curves (ROC) were generated using R (R Foundation for Statistical Computing, Vienna, Austria). Reference levels used are those described in Example I.

TABLE 12
Diagnostic Sensitivity and Specificity for salivary β-endorphin (A), chromogranin A (B), Annexin (C), BDNF (D), 3-methyl-2-oxobutyrate
(E), creatine (F), tyrosine (G), arginine (H), dihomo-linolenate (I) and linolenate (J) in individuals with sleep disorders.

| Parameters | A | AB | ABC | ABCD | ABCDE | ABCD EF | ABCDEF FG | ABCDE FGH | ABCDE FGHI | ABCDEFGHIJ |
|---|---|---|---|---|---|---|---|---|---|---|
| ROC AUC | 0.78 | 0.85 | 0.90 | 0.93 | 0.94 | 0.95 | 0.96 | 0.96 | 0.96 | 0.96 |
| Sensitivity (%) | 86 | 88 | 89 | 90 | 90 | 91 | 91.3 | 92 | 93 | 93 |
| Specificity (%) | 87 | 88 | 88 | 89.3 | 90 | 90 | 90 | 90 | 90 | 90 |
| Test Accuracy (%) | 86 | 88 | 89 | 89 | 90 | 90 | 91 | 91 | 91.3 | 91.5 |
| Positive Predictive Value (%) | 87 | 88 | 89 | 90 | 91 | 91.2 | 91.4 | 91.5 | 91.5 | 92 |
| Negative Predictive Value (%) | 85 | 87 | 88 | 89 | 90 | 91 | 91.4 | 91.5 | 91.6 | 91.7 |

As shown in Table 12, the combination models of β-endorphin, chromogranin A, Annexin and BDNF have high diagnostic values for diagnosis of sleepiness as compared to a single biomarker.

### Example X

### Stability and Reproducibility of β-Endorphin, Chromogranin A, Annexin 1, BDNF, 3-Methyl-2-Oxobutyrate, Creatine, Tyrosine, Arginine, Dihomo-Linolenate and Linolenate Measurements

Samples were collected from 16 normal healthy and 29 sleepiness patients (total 45 subjects). The samples were taken randomly and labeled such that the laboratories could not identify the diagnosis of the individuals sampled. The reproducibility of blinded quality control replicates was analyzed using the coefficient of variation (CV), a commonly used statistic to describe laboratory technical error, and the effect of delayed sample processing on biomarker levels in frozen samples at −60° C. (at 10 hours, 20 days and 30 days) was determined by estimating the standard deviation of the quality control values, divided by the mean of these values multiplied by 100. Between person and within person variances were estimated from repeated participant sample measurements using a random effects model, with participant ID as the random variable. Furthermore, the reproducibility of salivary β-endorphin, chromogranin A, Annexin 1, BDNF, 3-methyl-2-oxobutyrate, creatine, tyrosine, arginine, dihomo-linolenate and linolenate levels was assessed over a 10 and 15 day period by taking samples at 10 days and 15 days without any treatment during the diagnostic process and comparing the results. To evaluate reproducibility, we calculated ICCs (Intraclass Correlation Coefficient) by dividing the between-person variance by the sum of the within- and between-person variances; 95% confidence intervals (CI) were also calculated. The between and within person CVs were determined by taking the square root of the between- and within-person variance components from the random effects mixed model on the In [log] transformed scale, with approximate estimates derived by the eta method (Rosner B. Fundamentals of biostatistics. Belmont, Calif.: Duxbury; 2006). An ICC of <0.40 indicates poor reproducibility, 0.40 to 0.8 indicates fair to good reproducibility, and more than 0.8 indicates excellent reproducibility.

TABLE 13
ICCs calculated for delayed analysis and processing
of frozen samples at 10 hours, 20 days and 30 days
for various salivary biomarkers in subjects.

| | No. of | | | |
|---|---|---|---|---|
| | Participants/ | | Between | |
| | Number of | With-Person | Person | ICC |
| | Time Points | CV (%) | CV (%) | (95% CI) |
| CgA | 45/3 | 1.3 | 2.3 | 0.92 |
| β-endorphin | 45/3 | 1.3 | 2.5 | 0.88 |
| BDNF | 45/3 | 1.4 | 2.1 | 0.93 |
| Annexin 1 | 45/3 | 1.1 | 2.3 | 0.94 |
| Arginine | 45/3 | 1.2 | 2.4 | 0.91 |
| Tyrosine | 45/3 | 1.4 | 2.4 | 0.91 |
| Creatine | 45/3 | 1.4 | 2.3 | 0.92 |
| 3-methyl- | 45/3 | 1.6 | 2.2 | 0.86 |
| 2oxobutyrat | | | | |
| Dihomo- | 45/3 | 1.2 | 2.4 | 0.91 |
| linolenate | | | | |
| Linolenate | 45/3 | 1.4 | 2.5 | 0.9 |

The ICCs for the range of salivary biomarkers were high (ICCs; 0.87-0.94), indicating good to excellent reproducibility and stability.

TABLE 14
ICCs calculated at various time duration
(Day 1, Day 5 and Day 10) in all subjects

| | No. of | | | |
|---|---|---|---|---|
| | Participants/ | | Between | ICC |
| | number of | Within Person | Person | (95% CI) |
| | time points | CV (%) | CV (%) | s |
| CgA | 45/3 | 1.5 | 3.1 | 0.89 |
| B-endorphin | 45/3 | 1.6 | 3.2 | 0.87 |
| BDNF | 45/3 | 1.6 | 3.1 | 0.87 |
| Annexin 1 | 45/3 | 1.5 | 3.2 | 0.88 |
| Arginine | 45/3 | 1.7 | 3.4 | 0.87 |
| Tyrosine | 45/3 | 1.6 | 3.5 | 0.87 |
| Creatine | 45/3 | 1.7 | 3.4 | 0.87 |
| 3-methyl-2- | 45/3 | 1.6 | 3.2 | 0.87 |
| oxobutyrate | | | | |
| Dihomo- | 45/3 | 1.8 | 3.1 | 0.87 |
| linolenate | | | | |
| Linolenate | 45/3 | 1.6 | 3.1 | 0.87 |

The ICCs for the range of salivary biomarkers were high, indicating good to excellent reproducibility and stability.

The examples set forth above are offered to give those of ordinary skill in the art a complete disclosure and description of how to make and use the embodiments of the compositions, systems and methods of the disclosure, and are not intended to limit the scope of what the inventors regard as their disclosure. Modifications of the above-described modes for carrying out the disclosure that are obvious to persons of skill in the art are intended to be within the scope of the following claims.

What is claimed is:

1. A method of diagnosing sleepiness, comprising:
(a) collecting a saliva sample from an individual suspected of suffering a sleep disorder;
(b) applying the saliva sample to a solid support on which at least two agents, each of which binds to a different single biomarker selected from arginine, creatine, dihomo-linoleate, tyrosine, beta-endorphin, chromogranin A, annexin I, BDNF, 3-methyl-1-oxo-butyrate, or linolenate have been affixed, and wherein each agent binds to the different single biomarker to form a measurable complex of a single biomarker;
(c) measuring the measurable complex of each of the different single biomarkers to create an individual biomarker profile; and
(d) comparing the individual biomarker profile to reference levels for each of the different single biomarkers, wherein the individual biomarker profile meeting two or more criteria in a group of criteria is indicative of the sleep disorder, the group of criteria consisting of:
an arginine reference level greater than about 0.32 μmol/ml,
a creatine reference level greater about 0.47 μmol/ml,
a dihomo-linoleate reference level greater than about 34 μmol/ml,
a tyrosine reference level greater than about 0.57 μmol/ml,
a beta-endorphin reference level greater than about 1490 pg/ml,
a BDNF reference level greater than about 488 pg/ml,
a 3-methyl-oxobutyrate reference level greater than about 52 μmol/ml,
a linoleate reference level greater than about 36 μmol/ml,
chromogranin A reference level greater than about 3.3 μmol/mg protein, and
an annexin I reference level greater than about 55 ng/ml.

2. The method of claim 1, wherein:
the arginine reference level is greater than about 0.32 and less than about 0.65 μmol/ml,
the creatine reference level is greater than about 0.48 and less than about 0.75 μmol/ml,
the dihomo-linoleate reference level is greater than about 35 and less than about 43 μmol/ml,
the tyrosine reference level is greater than about 0.58 to about 0.85 μmol/ml,
the beta-endorphin reference level is greater than about 1491 and less than about 2100 pg/ml,
the BDNF reference level is greater than about 502 and less than about 679 pg/ml,
the 3-methyl-oxobutyrate reference level is greater than about 53 and less than about 71.8 μmol/ml
the linoleate reference level is greater than about 37 and less than about 52 μmol/ml
the chromogranin A reference level is greater than about 3.4 and less than about 5.0 μmol/mg protein, and
the annexin I reference level is greater than about 6 and less than about 84 ng/ml.

3. The method of claim 1, wherein the saliva sample from the individual suspected of having the sleep disorder is collected within a same time of day window as saliva samples used to determine the reference levels.

4. The method of claim 1, wherein the saliva sample from the individual suspected of having the sleep disorder is collected in a same manner as saliva samples used to determine the reference levels.

5. The method of claim 1, wherein the saliva sample from the individual suspected of having the sleep disorder is tested for levels of biomarkers using a same type of assay as the assay used to determine the reference levels of the biomarkers.

6. The method of claim 1, wherein the saliva sample from the individual suspected of having the sleep disorder is collected in a same manner and within a same time of day window as saliva samples used to determine the reference levels of each of the different single biomarkers and is tested for levels of each of the different single biomarkers using a same type of assay as the assay used to determine the reference levels of each of the different single biomarkers.

7. The method of claim 1, wherein the at least two agents bind to β-endorphin, chromogranin A, Annexin and BDNF.

8. The method of claim 1, wherein the at least two agents bind to arginine, creatine, dihomo-linoleate, tyrosine, beta-endorphin, chromogranin A, annexin 1, BDNF, 3-methyl-1-oxo-butyrate, or linolenate.

9. A method of monitoring effectiveness of a treatment for a sleep disorder comprising:
(a) collecting a first saliva sample from an individual diagnosed with the sleep disorder;
(b) applying the first saliva sample to a first solid support on which a plurality of agents, which in combination bind to at least two different single biomarkers selected from selected from arginine, creatine, dihomo-linoleate, tyrosine, beta-endorphin, chromogranin A, annexin I, BDNF, 3-methyl-1-oxo-butyrate, or linolenate have been affixed;
wherein each agent binds to the different single biomarker to form a measurable complex of the each of the different single biomarkers;
(c) measuring the measurable complex of each of the different single biomarkers selected from arginine, creatine, dihomo-linoleate, tyrosine, beta-endorphin, chromogranin A, annexin I, BDNF, 3-methyl-1-oxo-butyrate, or linolenate in the first saliva sample to create a first individual biomarker profile;
(d) treating the individual for the sleep disorder;
(e) collecting a second saliva sample from the individual diagnosed with the sleep disorder at a time point after the treatment for the sleep disorder has begun;
(f) applying the second saliva sample to a second solid support on which the plurality of agents which bind to the different single biomarker on the first solid support have been affixed to form a measurable complex of each of the different single biomarkers;
(g) measuring levels of each of the different single biomarkers selected from arginine, creatine, dihomo-linoleate, tyrosine, beta-endorphin, chromogranin A, annexin I, BDNF, 3-methyl-1-oxo-butyrate, or linolenate in the second saliva sample to create a second individual biomarker profile; and
(h) comparing the first individual biomarker profile and the second individual biomarker profile, wherein a decrease in levels of arginine, creatine, dihomo-linoleate, tyrosine, beta-endorphin, chromogranin A, annexin I, BDNF, 3-methyl-1-oxo-butyrate, or linolenate in the second individual biomarker profile relative to the first individual biomarker profile indicates that the treatment is effective.

10. The method of claim 9, wherein the first saliva sample for the first individual biomarker profile and the second saliva sample for the second individual biomarker profile are taken within a same time of day window.

11. The method of claim 9, wherein the first saliva sample for the first individual biomarker profile and the second saliva sample for the second individual biomarker profile are collected in a same manner.

12. The method of claim 9, wherein the first saliva sample for the first individual biomarker profile and the second saliva sample for the second individual biomarker profile are tested using a same type of assay.

13. The method of claim 9, wherein the saliva sample for the first individual biomarker profile and the saliva sample for the second individual biomarker profile are collected in a same manner and within a same time of day window and are tested using a same type of assay.

14. The method of claim 9, wherein the at least two biomarkers are β-endorphin, chromogranin A, Annexin and BDNF.

15. A kit for determining whether a patient has a sleep disorder comprising:
a plurality of test strips, each configured to produce a fluorescence level proportional to an amount present on one of the plurality of test strips of at least two biomarkers selected from arginine, creatine, dihomo-linoleate, tyrosine, beta-endorphin, chromogranin A, annexin 1, BDNF, 3-methyl-1-oxo-butyrate, or linolenate; and
a reading device configured to read the fluorescence level proportional to the level of each biomarker on each of the plurality of test strips after each of the plurality of test strips are exposed to a saliva sample and wherein when the fluorescence levels on the plurality of test strips indicate that two or more of the biomarkers meet two or more criteria in a group of criteria, the group of criteria consisting of:
an arginine reference level greater than about 0.32 μmol/ml,
a creatine reference level greater about 0.47 μmol/ml,
a dihomo-linoleate reference level greater than about 34 μmol/ml,
a tyrosine reference level greater than about 0.57 μmol/ml,
a beta-endorphin reference level greater than about 1490 pg/ml,
a BDNF reference level greater than about 488 pg/ml,
a 3-methyl-oxobutyrate reference level greater than about 52 μmol/ml,
a linoleate reference level greater than about 36 μmol/ml,
a chromogranin A reference level greater than about 3.3 μmol/mg protein, and
an annexin I reference level greater than about 55 ng/ml.

16. The kit of claim 15, wherein,
the arginine reference level is greater than about 0.32 and less than about 0.65 μmol/ml,
the creatine reference level is greater than about 0.48 and less than about 0.75 μmol/ml,
the dihomo-linoleate reference level is greater than about 35 and less than about 43 μmol/ml,
the tyrosine reference level is greater than about 0.58 to about 0.85 μmol/ml,
the beta-endorphin reference level is greater than about 1491 and less than about 2100 pg/ml,
the BDNF reference level is greater than about 502 and less than about 679 pg/ml,
the 3-methyl-oxobutyrate reference level is greater than about 53 and less than about 71.8 μmol/ml
the linoleate reference level is greater than about 37 and less than about 52 μmol/ml
the chromogranin A reference level is greater than about 3.4 and less than about 5.0 μmol/mg protein, and
the annexin I reference level is greater than about 6 and less than about 84 ng/ml.

17. The kit of claim 15, further comprising:
instructions to take the saliva sample from the patient in a same manner as saliva samples used to determine the reference levels.

18. The kit of claim 15, further comprising:
instructions to take the saliva sample from the patient within a same time of day window as saliva samples used to determine the reference levels.

19. The kit of claim 15, further comprising:
instructions to take the saliva sample from the patient in a same manner and within a same time of day window as saliva samples used to determine the reference levels.

20. The kit of claim 15, wherein the reference levels are determined from reference saliva samples, wherein the reference saliva samples are collected from one or more persons with a sleep disorder.
